(19) United States
(12) Patent Application Publication
(10) Pub. No.: US 2019/0011368 A1
(43) Pub. Date: Jan. 10, 2019

(54) QUANTITATIVE FLAGELLAR FLUORESCENT MARKERS AND STANDARDS

(71) Applicant: Marquette University, Milwaukee, WI (US)

(72) Inventors: Pinfen Yang, Shorewood, WI (US); Yi Liu, San Francisco, CA (US)

(73) Assignee: Marquette University, Milwaukee, WI (US)

(21) Appl. No.: 16/139,508

(22) Filed: Sep. 24, 2018

Related U.S. Application Data
(63) Continuation-in-part of application No. PCT/US2017/024051, filed on Mar. 24, 2017.
(60) Provisional application No. 62/312,772, filed on Mar. 24, 2016.

(51) Int. Cl. G01N 21/64

(52) U.S. Cl. CPC G01N 21/6458; C07K 17/14; C07K 2319/60; G01N 21/6428

(57) ABSTRACT

Disclosed are fluorescent markers that include a known number of copies of a fluorescently-labeled protein regularly interspersed along the length of the fluorescent marker. The fluorescent markers may be used to quantify fluorescently-labeled samples in fluorescent microscopy.

### CROSS-REFERENCE TO RELATED PATENT APPLICATIONS

The present application is a continuation-in-part application of International Application No. PCT/US2017/0240511, filed on Mar. 24, 2017, and published on Sep. 28, 2017 as WO 2017/165788, which application claims the benefit of priority under 35 U.S.C. § 119(e) to U.S. Provision Application No. 62/312,772, filed on Mar. 24, 2016, the contents of which are incorporated herein by reference in their entireties.

### BACKGROUND

The present invention relates to fluorescent markers and standards which may be useful in fluorescence microscopy. In particular, the invention relates to fluorescently-labeled flagella that may be useful in fluorescence microscopy

Biological molecule markers are a valuable tool in a variety of biological methods. For example, DNA or protein markers are indispensable for estimating the size and abundance of respective molecules in gel electrophoresis, a common procedure in biomedical science. Unfortunately, there are no equivalent markers that could be used easily to estimate the number of molecules of interests when performing fluorescence microscopy, which is a very common methodology used in research and diagnosis.

Here, we disclose fluorescent flagella that may be used as biological molecule markers in fluorescence microscopy. The fluorescent flagella typically include a recombinant fluorescent protein that is present in the fluorescent flagella at a known periodicity, in other words, with a known number of recombinant fluorescent proteins per unit length of the flagella. As such, the recombinant fluorescent protein has a known stoichiometry within the fluorescent flagella such that the fluorescence from the flagella can be measured and the relative fluorescence intensity per recombinant fluorescent protein can be determined. By comparing the fluorescence of the fluorescent flagella to the fluorescence of fluorescently-labeled sample molecules, the number of fluorescently-labeled sample molecules can be quantified rather easily. As such, the disclosed fluorescent flagella are useful as marker standards in fluorescent microscopy.

### SUMMARY

Disclosed are fluorescent markers. The disclosed fluorescent markers may be utilized in fluorescent microscopy in order to quantify a fluorescently-labeled sample or otherwise assess a fluorescently-labeled sample.

The disclosed fluorescent markers typically comprise a tubular or cylindrical biological structure, which has dimensions that make the fluorescent markers suitable for use in fluorescence microscopy. The biological structures of the fluorescent markers may include, but are not limited to proteinaceous microtubules or a macrostructure comprising proteinaceous microtubules such as a doublet microtubule, an axoneme, or a flagellum (e.g., eukaryotic flagellum).

The biological structure of the disclosed fluorescent markers typically is formed by multiple copies of at least one structural protein (SP). For example, the multiple copies of the structural protein may associate or assemble with each other non-covalently to form the biological structure, which may have a helical conformation. Suitable proteins forming the biological structure may include structural and non-structural proteins such as tubulin proteins, for example, as α-tubulin, β-tubulin, or a combination thereof such as a heterodimer.

The biological structure of the fluorescent markers comprises multiple copies of a fluorescently-labeled protein (FP). The fluorescent proteins are regularly interspersed along the length of the biological structure, and as such, the fluorescent proteins can be said to exhibit periodicity in the biological structure. Because the fluorescent proteins are regularly interspersed along the length of the biological structure, the biological structure has a known stoichiometry of fluorescent proteins per unit length of the biological structure and by measuring the length of the biological structure, the number of fluorescent proteins present in the structure can be estimated. Furthermore, the fluorescence intensity of the fluorescent marker can be measured and the intensity/per fluorescent protein can be calculated.

The fluorescently-labeled protein may comprise, consist essentially of, or consist of a fusion protein comprising a fluorescent protein portion and portion that associates with or assembles the fusion protein in the biological structure. The portion of the fusion protein that associates with or assembles the fusion protein in the biological structure may be referred to as an anchor portion of the fusion protein where this anchor portion anchors the fluorescent protein portion to the biological structure. The fluorescent protein portion is fused to the anchor portion, either directly or via a peptide linker, and the fluorescent protein portion may be fused to the C-terminus, the N-terminus, or any location of the anchor portion.

Suitable proteins or variants thereof for the fusion protein of the biological structure, for example as anchor portions of the fusion proteins, may include the radial spoke (RS) protein associated with a microtubule or a variant thereof, for example, where the biological structure is a microtubule or macrostructure comprising microtubules and doublet microtubules such as an axoneme or flagellum. Suitable RS proteins may include radial spoke protein 3 (RSP3). Suitable proteins or variants thereof for the fusion protein of the biological structure, for example as fluorescent protein portions of the fusion proteins, may include but are not limited to green fluorescent protein (GFP), enhanced green fluorescent protein (EGFP), mNeonGreen protein (NG), enhanced blue fluorescent protein (EBFP), mCherry fluorescent protein, tdTomato fluorescent protein, enhanced cyan fluorescent protein (ECFP), Midoriishi-Cyan1 protein, AmCyan1 protein, Azami-Green protein, mAzami-Green1 protein, ZsGreen1, enhanced yellow fluorescent protein (EYFP), Venus protein, ZsYellow protein, Kusabira-Orange1 protein, and mKusabira-Orange1 protein. As indicated, the fusion proteins disclosed herein may comprise the amino acid sequence of a radial spoke protein (RSP) of a flagellum or a variant thereof fused to the amino acid sequence of a fluorescent protein or variant thereof.

Instead of a fluorescent protein portion, the disclosed fusion proteins may comprise an anchor portion fused to an adapter protein or a portion of an adapter protein (i.e., and “adapter portion”) where the adapter portion of the fusion protein binds to a fluorophore label. Suitable adapter proteins may include biotinylated polypeptides that bind to streptavidin-conjugated fluorophore label, which may include non-protein fluorophore labels. As such, the disclosed fluorescently-labelled fusion proteins may comprise an anchor portion fused to biotinylated adapter polypeptide which binds to a streptavidin-conjugated fluorophore label.

Also contemplated herein are polynucleotides encoding the amino acid sequence of the fusion proteins disclosed herein. The polynucleotides may be operably linked to a promoter, for example within an expression vector. Also contemplated are isolated cells comprising expression vectors that express the fusion proteins. The isolated cells may be cultured in order to produce the fusion proteins and/or biological structures comprising the fusion proteins, for example where the fluorescent markers disclosed herein comprise the biological structures.

The disclosed fluorescent markers or fragments thereof optionally may be immobilized on a solid substrate, for example, a microscopic slide, which may be utilized in fluorescent microscopy. As such, also contemplated herein are methods for performing fluorescence microscopy. The methods utilize the fluorescent markers disclosed herein and may include a step of detecting fluorescence from the fluorescent marker or from a solid substrate having the fluorescent marker immobilized thereon while performing fluorescence microscopy and/or imaging the fluorescent marker.

In the disclosed methods for performing fluorescence microscopy, the disclosed fluorescent markers may be applied to a solid substrate such as a microscopic slide. Subsequently, a fluorescently-labeled sample may be applied to the slide prior to performing fluorescence microscopy. Fluorescence then may be detected from the fluorescent marker and/or the fluorescent marker may be imaged. Then, either concurrently or non-concurrently, fluorescence may be detected from the fluorescently-labeled sample and/or the fluorescently-labeled sample may be imaged, while performing fluorescence microscopy. In the methods for performing fluorescence microscopy, the fluorescent label of the marker may be the same as or different than the fluorescent label of the sample. The fluorescent marker may be imaged separated from the fluorescently-labeled sample and/or the fluorescent marker may be imaged together with the fluorescently-labeled sample.

### BRIEF DESCRIPTION OF THE FIGURES

FIG. 1. The 9+2 axoneme in Chlamydomonas flagella. (A, B) Cross and longitudinal sections of an axoneme. Radial spokes (white arrowhead) are anchored to each of the 9 outer doublets, and appear as a pair every 96 nm. (C) Each radial spoke contains two RSP3 with the C-termini near the spoke head region. The digital renditions of a 96 nm repeat were derived from cryo-electron tomograms of flagella with RSP3 (left, EM database ID, 5845; Oda et al., 2014) and with RSP3-streptavidin (right, EM database ID, 5847). Arrows, streptavidin tags. Bar, 100 nm.

FIG. 2A, FIG. 2B, and FIG. 2C. RSP3-NG flagella are brighter than RSP3-GFP flagella. (FIG. 2A) Western blot analysis of RSP3-FPs abundance in flagella. Flagella samples were harvested from wild type (WT), pf14 (RSP3 mutant), and pf14 cells expressing RSP3-NG or RSP3-GFP transgenes. The blots were probed for RSP3 and IC78, an outer dynein arm subunit as a loading control. (FIG. 2B) Live cell fluorescence microscopy of RSP3-NG (left) and RSP3-GFP (right) transgenic cells. Arrows, flagella. (FIG. 2C) Fluorescence intensity comparison of RSP3-NG and RSP3-GFP flagella in an image without (upper panel) and with background subtraction. The cells were mixed together prior to microscopy. Intensities of representative areas (arrows) were measured and plotted across the indicated region as intensity profiles. Bar, 10 μm.

FIG. 3A and FIG. 3B. Quantification of fluorescence intensity of flagella with RSP3-NG. (FIG. 3A) Individual RSP3-NG flagella had similar intensity. Each flagellum was measured across the middle region (upper panel) using the imageJ program. Relative intensities were plotted into a chart (lower panel, left). The average of the highest intensity was presented in a histogram (lower panel, right). (FIG. 3B) Overlapped regions were nearly twice as intense as non-overlapped regions. The area across overlapped (arrows, upper panel) regions and the nearby non-overlapped region were measured. The peak intensities (lower left panel) and the averages (lower right panel) were plotted into a histogram. Gray, overlapped regions; blue, non-overlapped region. Bar, 10 μm.

FIG. 4A and FIG. 4B. Fluorescence intensities of outer doublets with RSP3-NG. (FIG. 4A) Splayed RSP3-NG flagella adhered to the poly-L-lysine-coated slide. Splaying was induced by shear forces from gently moving the cover slip back-and-forth (upper panel). A partial splayed flagellum (the boxed area) was enlarged (lower panel). Three regions marked by color lines were measured. Red, three sub-fibers; orange, two sub-fibers; blue, an intact region (middle panel). Relative intensities are measured and presented as a profile plot (lower panel). (FIG. 4B) Fluorescent particles generated by shearing of unattached RSP3-NG flagella (upper panel). A partially splayed flagellum and fragmented particles (the boxed area) were enlarged (middle panel). Relative intensities at three regions were measured and plotted (lower panel). Blue arrow, intact region; red arrow, a splayed subfiber; green arrows, small particles. Bars, 5 μm.

FIG. 5A and FIG. 5B. NG retained fluorescence albeit with reduced intensity following methanol fixation. (FIG. 5A) RSP3-NG flagella with or without methanol treatments. RSP3-NG flagella immobilized on poly-L-lysine-coated slide were fixed with −20° C. methanol first. The fluorescent image was taken following rehydration and addition of unfixed flagella (upper panel). Relative intensities (middle panel) and the averages (lower panel) of areas were measured. Blue, unfixed; red, fixed; arrow and gray, overlapped region in fixed flagella. (FIG. 5B) WT cells expressing EB1-NG. Cells immobilized on poly-L-lysine-coated slides were fixed with methanol first. The fluorescent image was taken after rehydration and addition of live cells (left panels). EB1-NG comets had a similar punctate head in fixed cells (orange arrow) regardless of 1- or 10-second exposures. In contrast, comets in the live cell (blue arrow) appeared longer after a 10-second exposure because of elongation of growing microtubules. Relative intensities of the marked comets were measured (right panels). Bar, 10 μm.

FIG. 6A, FIG. 6B, FIG. 6C, and FIG. 6D. Applications of RSP3-NG flagella as fluorescence intensity standard. (FIG. 6A and FIG. 6B) Comparisons of RSP3-NG flagella with EB1-NG at the tip of flagella and with EB1-NG comets in the cell body. Cells expression RSP3-NG or EB1-NG were adhered to the glass slide to image the fluorescence in flagella at the same focal planes (FIG. 6A). For measuring comets in the cell body, EB1-NG cells were mixed with isolated RSP3-NG flagella before image acquisition (FIG. 6B). The corresponding intensity measurements were plotted in the right panels. Blue, RSP3-NG; red, EB1-NG. (FIG. 6C and FIG. 6D) Comparing of isolated RSP3-NG flagella with yeast strains expressing COX4-GFP targeted to mitochondria or Sis1-GFP in the cytosol. COX4-GFP decorated mitochondrial tubes (green and red arrows in C). The fluorescence intensity profiles showed that the intensity of Cox4-GFP was similar to that of RSP3-NG flagella for one cell, and more than 2× brighter for the other. A fraction of Sis1-GFP was enriched into a spot (red circle in D). The mean intensity (total intensities/area of a selected region) of the spots (red circle) was compared with that of 2-μm segments of 10 RSP3-NG flagella (blue rectangle). The averages of the peak intensity were plotted into a histogram.

FIG. 7. Diversifications of fluorescent flagella. The current fluorescent flagella are from algal strains expressing RSP3-GFP or RSP3-NeonGreen. For diversification, one way is to switch to fluorescent protein of different colors, such as mCherry or tdTomato; or to switch to SNAP-tag protein, which could be conjugated to fluorescent compounds, like Alexa 488, via chemical reactions. The current DNA construct was designed for easy switch of protein tags. SNAP-tag will allow customers to create their own standards. Alternatively, RSP3, the fluorescence carrier, could be switched to different flagellar proteins. This will allow us to produce flagella that are brighter or have at least two.

### DETAILED DESCRIPTION

The subject matter disclosed herein is described using several definitions, as set forth below and throughout the application.

Unless otherwise noted, the terms used herein are to be understood according to conventional usage by those of ordinary skill in the relevant art. In addition to the definitions of terms provided below, it is to be understood that as used in the specification, embodiments, and in the claims, the terms “a”, “an”, and “the” can mean one or more, depending upon the context in which the terms are used. For example, the term “a flagella” should be interpreted to mean “one or more flagella.”

As used herein, “about,” “approximately,” “substantially,” and “significantly” will be understood by persons of ordinary skill in the art and will vary to some extent on the context in which they are used. If there are uses of these terms which are not clear to persons of ordinary skill in the art given the context in which they are used, “about” and “approximately” will mean plus or minus ≤10% of the particular term and “substantially” and “significantly” will mean plus or minus >10% of the particular term.

As used herein, the terms “include” and “including” have the same meaning as the terms “comprise” and “comprising.” The terms “comprise” and “comprising” should be interpreted as being “open” transitional terms that permit the inclusion of additional components further to those components recited in the claims. The terms “consist” and “consisting of” should be interpreted as being “closed” transitional terms that do not permit the inclusion of additional components other than the components recited in the claims. The term “consisting essentially of” should be interpreted to be partially closed and allowing the inclusion only of additional components that do not fundamentally alter the nature of the claimed subject matter.

As used herein, the term “protein,” “polypeptide,” and “peptide” refer to biological molecules that include a polymer of amino acid residues joined by amide linkages. The term “amino acid residue,” includes but is not limited to amino acid residues contained in the group consisting of alanine (Ala or A), cysteine (Cys or C), aspartic acid (Asp or D), glutamic acid (Glu or E), phenylalanine (Phe or F), glycine (Gly or G), histidine (His or H), isoleucine (Ile or I), lysine (Lys or K), leucine (Leu or L), methionine (Met or M), asparagine (Asn or N), proline (Pro or P), glutamine (Gln or Q), arginine (Arg or R), serine (Ser or S), threonine (Thr or T), valine (Val or V), tryptophan (Trp or W), and tyrosine (Tyr or Y) residues. The term “amino acid residue” also may include amino acid residues contained in the group consisting of homocysteine, 2-Aminoadipic acid, N-Ethylasparagine, 3-Aminoadipic acid, Hydroxylysine, β-alanine, β-Amino-propionic acid, allo-Hydroxylysine acid, 2-Aminobutyric acid, 3-Hydroxyproline, 4-Aminobutyric acid, 4-Hydroxyproline, piperidinic acid, 6-Aminocaproic acid, Isodesmosine, 2-Aminoheptanoic acid, allo-Isoleucine, 2-Aminoisobutyric acid, N-Methylglycine, sarcosine, 3-Aminoisobutyric acid, N-Methylisoleucine, 2-Aminopimelic acid, 6-N-Methyllysine, 2,4-Diaminobutyric acid, N-Methylvaline, Desmosine, Norvaline, 2,2′-Diaminopimelic acid, Norleucine, 2,3-Diaminopropionic acid, Ornithine, and N-Ethylglycine. Typically, the amide linkages of the peptides are formed from an amino group of the backbone of one amino acid and a carboxyl group of the backbone of another amino acid.

As used herein, a “protein” or “polypeptide” is defined as a relatively long polymer of amino acids relative to a “peptide.” A protein or polypeptide typically has an amino acid length of greater than 50, 60, 70, 80, 90, or 100 amino acids, whereas a “peptide” is defined as a short polymer of amino acids, of a length typically of 50, 40, 30, 20 or less amino acids (Garrett & Grisham, Biochemistry, 2^{nd} edition, 1999, Brooks/Cole, 110).

A protein, polypeptide, or peptide as contemplated herein may be further modified to include non-amino acid moieties. Modifications may include but are not limited to acylation (e.g., O-acylation (esters), N-acylation (amides), S-acylation (thioesters)), acetylation (e.g., the addition of an acetyl group, either at the N-terminus of the protein or at lysine residues), formylation lipoylation (e.g., attachment of a lipoate, a C8 functional group), myristoylation (e.g., attachment of myristate, a C14 saturated acid), palmitoylation (e.g., attachment of palmitate, a C16 saturated acid), alkylation (e.g., the addition of an alkyl group, such as an methyl at a lysine or arginine residue), isoprenylation or prenylation (e.g., the addition of an isoprenoid group such as farnesol or geranylgeraniol), amidation at C-terminus, glycosylation (e.g., the addition of a glycosyl group to either asparagine, hydroxylysine, serine, or threonine, resulting in a glycoprotein). Distinct from glycation, which is regarded as a nonenzymatic attachment of sugars, polysialylation (e.g., the addition of polysialic acid), glypiation (e.g., glycosylphosphatidylinositol (GPI) anchor formation, hydroxylation, iodination (e.g., of thyroid hormones), and phosphorylation (e.g., the addition of a phosphate group, usually to serine, tyrosine, threonine or histidine).

Variants of the disclosed proteins, polypeptide, and peptides also are contemplated herein. As used herein, a “variant” refers to a protein, polypeptide, or peptide molecule having an amino acid sequence that differs from a reference protein, polypeptide, or peptide molecule. A variant may have one or more insertions, deletions, or substitutions of an amino acid residue relative to a reference protein, polypeptide, or peptide. A variant may include a fragment of a reference protein, polypeptide, or peptide. For example, reference proteins, polypeptides, or peptides may comprise, consist essentially of, or consist of any of the amino acid sequence of SEQ ID NOs:1-7). A RSP3 variant molecule has one or more insertions, deletions, or substitution of at least one amino acid residue relative to the RSP3 full-length polypeptide, which is presented as SEQ ID NO: 1.

A “deletion” refers to a change in the amino acid or that results in the absence of one or more amino acid residues relative to a reference protein, polypeptide, or peptide. A deletion removes at least 1, 2, 3, 4, 5, 10, 20, 50, 100, 200, or more amino acids residues relative to a reference protein, polypeptide, or peptide. A deletion may include an internal deletion or a terminal deletion (e.g., an N-terminal truncation, a C-terminal truncation or both of a reference polypeptide). A “variant” of a reference polypeptide sequence may include a deletion relative to the reference polypeptide sequence.

A “fragment” is a portion of an amino acid sequence which is identical in sequence to but shorter in length than a reference sequence. A fragment may comprise up to the entire length of the reference sequence, minus at least one amino acid residue. For example, a fragment may comprise from 5 to 1000 contiguous amino acid residues of a reference polypeptide, respectively. In some embodiments, a fragment may comprise at least 5, 10, 15, 20, 25, 30, 40, 50, 60, 70, 80, 90, 100, 150, 250, or 500 contiguous amino acid residues of a reference polypeptide. Fragments may be preferentially selected from certain regions of a molecule. The term “at least a fragment” encompasses the full length polypeptide. A fragment may include an N-terminal truncation, a C-terminal truncation, or both relative to full-length (i.e., relative to any of SEQ ID NOs:1-7). A fragment of RSP3 may comprise or consist essentially of a contiguous amino acid sequence of RSP3. A “variant” of a reference polypeptide sequence may include a fragment of the reference polypeptide sequence.

The words “insertion” and “addition” refer to changes in an amino acid sequence resulting in the addition of one or more amino acid residues. An insertion or addition may refer to 1, 2, 3, 4, 5, 10, 20, 30, 40, 50, 60, 70, 80, 90, 100, 150, or 200 amino acid residues or a range of amino acid residues bounded by any of these values (e.g., an insertion or addition of 5-10 amino acids). A “variant” of a reference polypeptide sequence may include an insertion or addition relative to the reference polypeptide sequence.

Fusion proteins also are contemplated herein. A “fusion protein” refers to a protein formed by the fusion of at least one first protein as disclosed herein (e.g., RSP3 or a variant thereof) to at least one molecule of a second, heterologous protein or a variant thereof as disclosed herein (e.g., GFP or NG). The heterologous protein(s) may be fused at the N-terminus, the C-terminus, or both termini of the first protein. A fusion protein comprises at least a fragment or variant of the first protein and at least a fragment or variant of the second, heterologous protein, which are associated with one another, preferably by genetic fusion (i.e., the fusion protein is generated by translation of a nucleic acid in which a polynucleotide encoding all or a portion of the first protein is joined in-frame with a polynucleotide encoding all or a portion of the second, heterologous protein). The first protein and second, heterologous protein, once part of the fusion protein, may each be referred to herein as a “portion”, “region” or “moiety” of the fusion protein (e.g., a “a protein portion,” which may include RSP3 or a variant thereof, or a “second, heterologous protein portion,” which may include a fluorescent protein or a variant thereof).

“Homology” refers to sequence similarity or, interchangeably, sequence identity, between two or more polypeptide sequences. Homology, sequence similarity, and percentage sequence identity may be determined using methods in the art and described herein.

The phrases “percent identity” and “% identity,” as applied to polypeptide sequences, refer to the percentage of residue matches between at least two polypeptide sequences aligned using a standardized algorithm. Methods of polypeptide sequence alignment are well-known. Some alignment methods take into account conservative amino acid substitutions. Such conservative substitutions, explained in more detail above, generally preserve the charge and hydrophobicity at the site of substitution, thus preserving the structure (and therefore function) of the polypeptide. Percent identity for amino acid sequences may be determined as understood in the art. (See, e.g., U.S. Pat. No. 7,396,664, which is incorporated herein by reference in its entirety). A suite of commonly used and freely available sequence comparison algorithms is provided by the National Center for Biotechnology Information (NCBI) Basic Local Alignment Search Tool (BLAST) (Altschul, S. F. et al. (1990) J. Mol. Biol. 215:403 410), which is available from several sources, including the NCBI, Bethesda, Md., at its website. The BLAST software suite includes various sequence analysis programs including “blastp,” that is used to align a known amino acid sequence with other amino acids sequences from a variety of databases.

Percent identity may be measured over the length of an entire defined polypeptide sequence, for example, as defined by a particular SEQ ID number, or may be measured over a shorter length, for example, over the length of a fragment taken from a larger, defined polypeptide sequence, for instance, a fragment of at least 15, at least 20, at least 30, at least 40, at least 50, at least 70 or at least 150 contiguous residues. Such lengths are exemplary only, and it is understood that any fragment length supported by the sequences shown herein, in the tables, figures or Sequence Listing, may be used to describe a length over which percentage identity may be measured.

A “variant” of a particular polypeptide sequence may be defined as a polypeptide sequence having at least 50% sequence identity to the particular polypeptide sequence over a certain length of one of the polypeptide sequences using blastp with the “BLAST 2 Sequences” tool available at the National Center for Biotechnology Information's website. (See Tatiana A. Tatusova, Thomas L. Madden (1999), “Blast 2 sequences—a new tool for comparing protein and nucleotide sequences”, FEMS Microbiol Lett. 174:247-250). Such a pair of polypeptides may show, for example, at least 60%, at least 70%, at least 80%, at least 90%, at least 91%, at least 92%, at least 93%, at least 94%, at least 95%, at least 96%, at least 97%, at least 98%, or at least 99% or greater sequence identity over a certain defined length of one of the polypeptides. A “variant” may have substantially the same functional activity as a reference polypeptide. For example, a variant may exhibit or more biological activities associated with PEDF. “Substantially isolated or purified” nucleic acid or amino acid sequences are contemplated herein. The term “substantially isolated or purified” refers to nucleic acid or amino acid sequences that are removed from their natural environment, and are at least 60% free, preferably at least 75% free, and more preferably at least 90% free, even more preferably at least 95% free from other components with which they are naturally associated.

The amino acid sequences or the proteins, polypeptide, and peptides contemplated herein may include conservative amino acid substitutions relative to a reference amino acid sequence. For example, a variant, mutant, or derivative peptide may include conservative amino acid substitutions relative to a reference molecule. “Conservative amino acid substitutions” are those substitutions that are a substitution of an amino acid for a different amino acid where the substitution is predicted to interfere least with the properties of the reference polypeptide. In other words, conservative amino acid substitutions substantially conserve the structure and the function of the reference polypeptide. The following table provides a list of exemplary conservative amino acid substitutions which are contemplated herein:

| Original Residue | Conservative Substitution |
|---|---|
| Ala | Gly, Ser |
| Arg | His, Lys |
| Asn | Asp, Gln, His |
| Asp | Asn, Glu |
| Cys | Ala, Ser |
| Gln | Asn, Glu, His |
| Glu | Asp, Gln, His |
| Gly | Ala |
| His | Asn, Arg, Gln, Glu |
| Ile | Leu, Val |
| Leu | Ile, Val |
| Lys | Arg, Gln, Glu |
| Met | Leu, Ile |
| Phe | His, Met, Leu, Trp, Tyr |
| Ser | Cys, Thr |
| Thr | Ser, Val |
| Trp | Phe, Tyr |
| Tyr | His, Phe, Trp |
| Val | Ile, Leu, Tyr |

Conservative amino acid substitutions generally maintain (a) the structure of the polypeptide backbone in the area of the substitution, for example, as a beta sheet or alpha helical conformation, (b) the charge or hydrophobicity of the molecule at the site of the substitution, and/or (c) the bulk of the side chain.

The disclosed proteins, polypeptide, peptides, or variants thereof may have one or functional or biological activities exhibited by a reference polypeptide (e.g., one or more functional or biological activities exhibited by RSP3). For example, a variant protein such as a fragment may exhibit one or more biological activities associated with a reference protein such as RSP3, GFP, or NG. A variant of RSP3 may exhibit one or more biological activities associated with RSP3, including, but not limited to dimerization and association with a microtubule and axoneme.

Also disclosed herein are polynucleotides, for example polynucleotide sequences that encode the polypeptides and proteins disclosed herein (e.g., DNA that encodes a polypeptide having the amino acid sequence of any of SEQ ID NOs:1-7 or DNA that encodes a polypeptide variant having an amino acid sequence with at least about 20%, 30%, 40%, 50%, 60%, 70%, 80%, 90%, 95%, 96%, 97%, 98%, or 99% sequence identity to any of SEQ ID NOs: 1-7).

The terms “polynucleotide,” “polynucleotide sequence,” “nucleic acid” and “nucleic acid sequence” refer to a nucleotide, oligonucleotide, polynucleotide (which terms may be used interchangeably), or any fragment thereof. These phrases also refer to DNA or RNA of genomic, natural, or synthetic origin (which may be single-stranded or double-stranded and may represent the sense or the antisense strand).

Regarding polynucleotide sequences, the terms “percent identity” and “% identity” refer to the percentage of residue matches between at least two polynucleotide sequences aligned using a standardized algorithm. Such an algorithm may insert, in a standardized and reproducible way, gaps in the sequences being compared in order to optimize alignment between two sequences, and therefore achieve a more meaningful comparison of the two sequences. Percent identity for a nucleic acid sequence may be determined as understood in the art. (See, e.g., U.S. Pat. No. 7,396,664, which is incorporated herein by reference in its entirety). A suite of commonly used and freely available sequence comparison algorithms is provided by the National Center for Biotechnology Information (NCBI) Basic Local Alignment Search Tool (BLAST), which is available from several sources, including the NCBI, Bethesda, Md., at its website. The BLAST software suite includes various sequence analysis programs including “blastn,” that is used to align a known polynucleotide sequence with other polynucleotide sequences from a variety of databases. Also available is a tool called “BLAST 2 Sequences” that is used for direct pairwise comparison of two nucleotide sequences. “BLAST 2 Sequences” can be accessed and used interactively at the NCBI website. The “BLAST 2 Sequences” tool can be used for both blastn and blastp (discussed above).

Regarding polynucleotide sequences, percent identity may be measured over the length of an entire defined polynucleotide sequence, for example, as defined by a particular SEQ ID number, or may be measured over a shorter length, for example, over the length of a fragment taken from a larger, defined sequence, for instance, a fragment of at least 20, at least 30, at least 40, at least 50, at least 70, at least 100, or at least 200 contiguous nucleotides. Such lengths are exemplary only, and it is understood that any fragment length supported by the sequences shown herein, in the tables, figures, or Sequence Listing, may be used to describe a length over which percentage identity may be measured.

Regarding polynucleotide sequences, “variant,” “mutant,” or “derivative” may be defined as a nucleic acid sequence having at least 50% sequence identity to the particular nucleic acid sequence over a certain length of one of the nucleic acid sequences using blastn with the “BLAST 2 Sequences” tool available at the National Center for Biotechnology Information's website. (See Tatiana A. Tatusova, Thomas L. Madden (1999), “Blast 2 sequences—a new tool for comparing protein and nucleotide sequences”, FEMS Microbiol Lett. 174:247-250). Such a pair of nucleic acids may show, for example, at least 20%, at least 30%, at least 40%, at least 50%, at least 60%, at least 70%, at least 80%, at least 85%, at least 90%, at least 91%, at least 92%, at least 93%, at least 94%, at least 95%, at least 96%, at least 97%, at least 98%, or at least 99% or greater sequence identity over a certain defined length.

Nucleic acid sequences that do not show a high degree of identity may nevertheless encode similar amino acid sequences due to the degeneracy of the genetic code where multiple codons may encode for a single amino acid. It is understood that changes in a nucleic acid sequence can be made using this degeneracy to produce multiple nucleic acid sequences that all encode substantially the same protein. For example, polynucleotide sequences as contemplated herein may encode a protein and may be codon-optimized for expression in a particular host. In the art, codon usage frequency tables have been prepared for a number of host organisms including humans, mouse, rat, pig, E. coli, plants, and other host cells.

A “recombinant nucleic acid” is a sequence that is not naturally occurring or has a sequence that is made by an artificial combination of two or more otherwise separated segments of sequence. This artificial combination is often accomplished by chemical synthesis or, more commonly, by the artificial manipulation of isolated segments of nucleic acids, e.g., by genetic engineering techniques known in the art. The term recombinant includes nucleic acids that have been altered solely by addition, substitution, or deletion of a portion of the nucleic acid. Frequently, a recombinant nucleic acid may include a nucleic acid sequence operably linked to a promoter sequence. Such a recombinant nucleic acid may be part of a vector that is used, for example, to transform a cell.

The nucleic acids disclosed herein may be “substantially isolated or purified.” The term “substantially isolated or purified” refers to a nucleic acid that is removed from its natural environment, and is at least 60% free, preferably at least 75% free, and more preferably at least 90% free, even more preferably at least 95% free from other components with which it is naturally associated.

“Transformation” or “transfected” describes a process by which exogenous nucleic acid (e.g., DNA or RNA) is introduced into a recipient cell. Transformation or transfection may occur under natural or artificial conditions according to various methods well known in the art, and may rely on any known method for the insertion of foreign nucleic acid sequences into a prokaryotic or eukaryotic host cell. The method for transformation or transfection is selected based on the type of host cell being transformed and may include, but is not limited to, bacteriophage or viral infection or non-viral delivery. Methods of non-viral delivery of nucleic acids include lipofection, nucleofection, microinjection, electroporation, heat shock, particle bombardment, biolistics, virosomes, liposomes, immunoliposomes, polycation or lipid:nucleic acid conjugates, naked DNA, artificial virions, and agent-enhanced uptake of DNA. Lipofection is described in e.g., U.S. Pat. Nos. 5,049,386, 4,946,787; and 4,897,355) and lipofection reagents are sold commercially (e.g., Transfectam™ and Lipofectin™). Cationic and neutral lipids that are suitable for efficient receptor-recognition lipofection of polynucleotides include those of Felgner, WO 91/17424; WO 91/16024. Delivery can be to cells (e.g. in vitro or ex vivo administration) or target tissues (e.g. in vivo administration). The term “transformed cells” or “transfected cells” includes stably transformed or transfected cells in which the inserted DNA is capable of replication either as an autonomously replicating plasmid or as part of the host chromosome, as well as transiently transformed or transfected cells which express the inserted DNA or RNA for limited periods of time.

The polynucleotide sequences contemplated herein may be present in expression vectors. For example, the vectors may comprise: (a) a polynucleotide encoding an ORF of a protein; (b) a polynucleotide that expresses an RNA that directs RNA-mediated binding, nicking, and/or cleaving of a target DNA sequence; and both (a) and (b). The polynucleotide present in the vector may be operably linked to a prokaryotic or eukaryotic promoter. “Operably linked” refers to the situation in which a first nucleic acid sequence is placed in a functional relationship with a second nucleic acid sequence. For instance, a promoter is operably linked to a coding sequence if the promoter affects the transcription or expression of the coding sequence. Operably linked DNA sequences may be in close proximity or contiguous and, where necessary to join two protein coding regions, in the same reading frame. Vectors contemplated herein may comprise a heterologous promoter (e.g., a eukaryotic or prokaryotic promoter) operably linked to a polynucleotide that encodes a protein. A “heterologous promoter” refers to a promoter that is not the native or endogenous promoter for the protein or RNA that is being expressed.

As used herein, “expression” refers to the process by which a polynucleotide is transcribed from a DNA template (such as into and mRNA or other RNA transcript) and/or the process by which a transcribed mRNA is subsequently translated into peptides, polypeptides, or proteins. Transcripts and encoded polypeptides may be collectively referred to as “gene product.” If the polynucleotide is derived from genomic DNA, expression may include splicing of the mRNA in a eukaryotic cell.

The term “vector” refers to some means by which nucleic acid (e.g., DNA) can be introduced into a host organism or host tissue. There are various types of vectors including plasmid vector, bacteriophage vectors, cosmid vectors, bacterial vectors, and viral vectors. As used herein, a “vector” may refer to a recombinant nucleic acid that has been engineered to express a heterologous polypeptide (e.g., the fusion proteins disclosed herein). The recombinant nucleic acid typically includes cis-acting elements for expression of the heterologous polypeptide.

Any of the conventional vectors used for expression in eukaryotic cells may be used for directly introducing DNA into a subject. Expression vectors containing regulatory elements from eukaryotic viruses may be used in eukaryotic expression vectors (e.g., vectors containing SV40, CMV, or retroviral promoters or enhancers). Exemplary vectors include those that express proteins under the direction of such promoters as the SV40 early promoter, SV40 later promoter, metallothionein promoter, human cytomegalovirus promoter, murine mammary tumor virus promoter, and Rous sarcoma virus promoter. Expression vectors as contemplated herein may include eukaryotic or prokaryotic control sequences that modulate expression of a heterologous protein (e.g. the fusion protein disclosed herein). Prokaryotic expression control sequences may include constitutive or inducible promoters (e.g., T3, T7, Lac, trp, or phoA), ribosome binding sites, or transcription terminators.

The vectors contemplated herein may be introduced and propagated in a prokaryote, which may be used to amplify copies of a vector to be introduced into a eukaryotic cell or as an intermediate vector in the production of a vector to be introduced into a eukaryotic cell (e.g. amplifying a plasmid as part of a viral vector packaging system). A prokaryote may be used to amplify copies of a vector and express one or more nucleic acids, such as to provide a source of one or more proteins for delivery to a host cell or host organism. Expression of proteins in prokaryotes may be performed using Escherichia coli with vectors containing constitutive or inducible promoters directing the expression of either a protein or a fusion protein comprising a protein or a fragment thereof. Fusion vectors add a number of amino acids to a protein encoded therein, such as to the amino terminus of the recombinant protein. Such fusion vectors may serve one or more purposes, such as: (i) to increase expression of recombinant protein; (ii) to increase the solubility of the recombinant protein; (iii) to aid in the purification of the recombinant protein by acting as a ligand in affinity purification (e.g., a His tag); (iv) to tag the recombinant protein for identification (e.g., such as Green fluorescence protein (GFP) or an antigen (e.g., HA) that can be recognized by a labeled antibody); (v) to promote localization of the recombinant protein to a specific area of the cell (e.g., where the protein is fused (e.g., at its N-terminus or C-terminus) to a nuclear localization signal (NLS) which may include the NLS of SV40, nucleoplasmin, C-myc, M9 domain of hnRNP A1, or a synthetic NLS). The importance of neutral and acidic amino acids in NLS have been studied. (See Makkerh et al. (1996) Curr Biol 6(8):1025-1027). Often, in fusion expression vectors, a proteolytic cleavage site is introduced at the junction of the fusion moiety and the recombinant protein to enable separation of the recombinant protein from the fusion moiety subsequent to purification of the fusion protein. Such enzymes, and their cognate recognition sequences, include Factor Xa, thrombin and enterokinase.

Quantitative Flagellar Fluorescent Markers and Standards

The following embodiments are illustrative and should not be interpreted to limit the scope of the claimed invention.

Disclosed are fluorescent markers. The disclosed fluorescent markers may be utilized in fluorescent microscopy in order to quantify a fluorescently-labeled sample or otherwise assess a fluorescently-labeled sample.

The disclosed fluorescent markers typically comprise a tubular or cylindrical biological structure. Typically, the biological structure of the fluorescent markers has dimensions that make the fluorescent markers suitable for use in fluorescence microscopy. In some embodiments, the biological structure has a length of less than about any of the following values: 2 mm, 1 mm, 0.5 mm, 0.2 mm, 0.1 mm, 0.05 mm, 0.02 mm, 0.01 mm, 0.005 mm, 0.002 mm or less; and/or the biological structure has a length of greater than about the following values: 0.001 mm, 0.002 mm, 0.005 mm, 0.01 mm, 0.02 mm, 0.05 mm, 0.1 mm, 0.2 mm, 0.5 mm, or 1 mm; or the biological structure may have a length within a range bounded by two of any of these values. The biological structure of the fluorescent markers typically has a length (L) that is significantly greater than its diameter (D), for example where the ratio L/D typically is greater than about the following values: 5, 10, 20, 30, 40, 50, 100, 200, 500, 1000 or greater, or the ratio L/D is within a range bounded by any two of these values. The biological structure of the markers typically has a diameter less than about the following values: 1000 nm 500 nm, 400 nm, 300 nm, 200 nm, 100 nm 50 nm 40, 30 nm, 20 nm, 10 nm; and/or the biological structure has a diameter greater than about 0.5 nm, 1 nm, 5 nm or greater: or the biological structure may have a length within a range bounded by any two of these values, for example between 20-30 nm or between 100-400 nm.

In some embodiments, the biological structure is a microtubule or a macrostructure comprising microtubules such as a doublet microtubule, an axoneme, or a flagellum, for example a eukaryotic flagellum. In the disclosed fluorescent markers, the dimensions of a microtubule may vary, but typically a microtubule of the disclosed markers has an outer diameter of less than about any of the following values: 100 nm, 50 nm, 40 nm, 30 nm, 20 nm or 10 nm; and/or the microtubule has an outer diameter greater than about any of the following values: 1 nm, 5 nm, 10 nm, 20 nm, 30 nm, 40 nm, or 50 nm; or the microtubule has an outer diameter within a range bounded by any two of these values, for example, 20-30 nm or approximately 24 nm. In the disclosed fluorescent markers, the microtubule typically has a length greater than about the following values: 0.001 mm, 0.002 mm, 0.005 mm, 0.01 mm, 0.02 mm, 0.05 mm, 0.1 mm, 0.2 mm, 0.5 mm, or 1 mm; and/or the microtubule may have a length less than about 2 mm, 1 mm, 0.5 mm, 0.2 mm, 0.1 mm, 0.05 mm, 0.02 mm, 0.01 mm, 0.005 mm, or 0.002 mm; or the microtubule may have a length within a range bounded by two of any of these values.

In some embodiments, the biological structure is a doublet microtubule comprising an A-microtubule and a B-microtubule as known in the art. In the disclosed fluorescent markers, the dimensions of a doublet microtubule may vary, but typically a doublet microtubule of the disclosed markers has an average effective outer diameter of less than about any of the following values: 100 nm, 50 nm, 40 nm, 30 nm, 20 nm or 10 nm; and/or the microtubule has an outer diameter greater than about any of the following values: 1 nm, 5 nm, 10 nm, 20 nm, 30 nm, 40 nm, or 50 nm; or the doublet microtubule has an average effective outer diameter within a range bounded by any two of these values, for example, 20-30 nm or approximately 24 nm. In the disclosed fluorescent markers, the doublet microtubule typically has a length greater than about the following values: 0.001 mm, 0.002 mm, 0.005 mm, 0.01 mm, 0.02 mm, 0.05 mm, 0.1 mm, 0.2 mm, 0.5 mm, or 1 mm; and/or the doublet microtubule may have a length less than about 2 mm, 1 mm, 0.5 mm, 0.2 mm, 0.1 mm, 0.05 mm, 0.02 mm, 0.01 mm, 0.005 mm, or 0.002 mm; or the microtubule may have a length within a range bounded by two of any of these values.

In some embodiments, the biological structure is an axoneme or a flagellum comprising an axoneme (e.g., an axoneme surrounded by a plasma membrane). An axoneme includes a 9+2 arrangement of microtubules and doublet microtubules as known in the art. In the disclosed fluorescent markers, the dimensions of an axoneme or flagellum may vary, but typically an axoneme or flagellum has diameter of greater than about any of the following values: 100 nm, 150 nm 200 nm, 250 nm, 300 nm, 350 nm, 400 nm, 450 nm, or 500 nm: and/or the axoneme or flagellum has a diameter less than about any of the following values: 500 nm, 450 nm, 400 nm, 350 nm, 300 nm, 250 nm, 200 nm, 150 nm, or 100 nm: or the axoneme or flagellum has a diameter within a range bounded by any two of these values, for example between 100-400 nm or about 250 nm. In the disclosed fluorescent markers, the axoneme or flagellum typically has a length greater than about the following values: 0.001 mm, 0.002 mm, 0.005 mm, 0.01 mm, 0.02 mm, 0.05 mm, 0.1 mm, 0.2 mm, 0.5 mm, or 1 mm; and/or the axoneme or flagellum may have a length less than about 2 mm, 1 mm, 0.5 mm, 0.2 mm, 0.1 mm, 0.05 mm, 0.02 mm, 0.01 mm, 0.005 mm, or 0.002 mm; or the axoneme or flagellum may have a length within a range bounded by two of any of these values.

The biological structure of the disclosed fluorescent markers typically is formed by multiple copies of at least one structural protein (SP). For example, the multiple copies of the structural protein may associate or assemble with each other non-covalently to form the biological structure. In some embodiments, the multiple copies of the structural protein are assembled in a helical conformation (e.g., having 13 copies of the structural protein per turn of the helix). Suitable structural protein s may include tubulin proteins such as α-tubulin, β-tubulin, or a combination thereof such as a heterodimer. The structural protein s may assemble to form a microtubule or a doublet microtubule. As such, the biological structure may comprise a microtubule or a doublet microtubule or a macrostructure comprising one or more microtubule or doublet microtubule, such as an axoneme having a 9+2 configuration of microtubules and double microtubules or a flagellum comprising the axoneme (e.g., an axoneme surrounded by a plasma membrane).

The biological structure of the fluorescent markers comprises multiple copies of a fluorescently-labeled protein (FP). The fluorescent proteins are regularly interspersed along the length of the biological structure, and as such, the fluorescent proteins can be said to exhibit periodicity in the biological structure. For example, the biological structure may comprise, consist essentially of, or consist of any number of fluorescent proteins selected from the following values: 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152, 154, 156, 158, 160, 162, 164, 166, 168, 170, 172, 174, 176, 178, 180, 182, 184, 186, 188, 190, 192, 194, 196, 198, or 200 fluorescent proteins per unit length of the biological structure (e.g., per ˜100 nm length of the biological structure, per 96 nm), or the biological structure may comprise, consist essentially of, or consist of any number of fluorescent proteins within a range bounded by any of two of these values per unit length of the biological structure. For example, in some embodiments the biological structure may have ˜2 fluorescent proteins per 96 nm length of the biological structure (e.g., where the biological structure is a microtubule), or the biological structure may have ˜4 fluorescent proteins per 96 nm length of the biological structure (e.g., where the biological structure is a doublet microtubule) or the biological structure may have ˜36 fluorescent proteins per 96 nm length of the biological structure (e.g., where the biological structure is an axoneme). Because the fluorescent proteins are regularly interspersed along the length of the biological structure, the biological structure has a known stoichiometry of fluorescent proteins per unit length of the biological structure and by measuring the length of the biological structure, the number of fluorescent proteins present in the structure can be estimated.

The fluorescently-labeled protein may comprise, consist essentially of, or consist of a fusion protein comprising a fluorescent protein portion and portion that associates with or assembles in the biological structure. The portion of the fusion protein that associates with or assembles in the biological structure may be referred to as an anchor portion of the fusion protein where this anchor portion anchors the fluorescent protein portion to the biological structure. The fluorescent protein portion may be fused to the N-terminus, the C-terminus, or any location of the anchor portion but typically the fluorescent protein portion is fused at the C-terminus of the anchor portion, either directly or via an amino acid linker of less than about 10, 9, 8, 7, 6, 5, 4, 3, 2, or 1 amino acid.

Suitable proteins or variants thereof for the fusion protein of the biological structure, for example as anchor portions of the fusion proteins, may include, but are not limited to, any protein that is regularly interspersed along the length of a microtubule, doublet microtubule, or axoneme. In some embodiments, the biological structure includes a fusion protein of a radial spoke protein (RS) associated with a microtubule or a variant thereof, for example, where the biological structure is a microtubule or macrostructure comprising microtubules and doublet microtubules such as an axoneme or flagellum. Suitable RS proteins may include radial spoke protein 3. The amino acid sequence of RSP3 of Chlamydomonas reinhardtii is provided as SEQ ID NO: 1. As such, suitable proteins for the fusion protein may include the amino acid sequence of SEQ ID NO:1 or variants thereof exhibiting at least about 50%, 60%, 70%, 80%, 90%, 95%, 96%, 97%, 98%, or 99% sequence identity, where preferably the protein or variant associates with of assembles in the biological structure.

Other suitable proteins for the fluorescent markers may include flagellar associated proteins such as flagellar-associated protein 20 (FAP20) (e.g., the sequence of Chlamydomonas reinhardtii FAP20 is provided as SEQ ID NO:8). For example. FAP20 is a feasible flagellar protein carrier (Yanagisawa et al., 2014) and is likely more abundant than RSP3. FAP20 may be utilized to create a fusion protein such as FAP20-NG and FAP20-mCherry (e.g., in strains of Chlamydomonas as described in the Example section below). It is possible that FAP20-NG flagella will be brighter than RSP3-NG flagella because FAP20 is more abundant in flagella than RSP3. FAP20-mCherry also may provide a red fluorescence standard. A FAP20-mCherry strain of organism (e.g., such as Chlamydomonas) may be crossed with s RSP3-NG strain to create double mutants. The double-tagged flagella with both RSP3-NG and FAP20-mCherry will serve as standards for dual labeling samples. Another product is RSP3-SNAP-tag flagella that may be converted into RSP3-SNAP-tag-guanine-Alexa 488 flagella as described below (e.g., where the fusion protein includes an adapter portion). The other suitable carriers include, but are not limited to, subunits of radial spokes (Oda et al., 2014), dynein motors (Hom et al., 2012), the central pair apparatus (Teves et al., 2016) and microtubule-associated complexes in the axoneme (e.g. King and Patel-King, 2015; Norrander et al., 2000). Similar strategies could be replicated in other ciliated organisms, such as Tetrahymena and Paramecium.

Suitable proteins or variants thereof for the fusion protein of the biological structure, for example as fluorescent protein portions of the fusion proteins, may include but are not limited to green fluorescent protein (GFP), enhanced green fluorescent protein (EGFP), mNeonGreen protein (NG), enhanced blue fluorescent protein (EBFP), mCherry fluorescent protein, tdTomato fluorescent protein, enhanced cyan fluorescent protein (ECFP), Midoriishi-Cyan1 protein, AmCyan1 protein, Azami-Green protein, mAzami-Green1 protein, ZsGreen1, enhanced yellow fluorescent protein (EYFP), Venus protein, ZsYellow protein, Kusabira-Orange1 protein, and mKusabira-Orange1 protein. (See Suzuki et al., “Recent Advanced in Fluorescent Labeling Techniques for Fluorescence Microscopy, Acta Histochem. Cytochem. 40(5):131-137, 2007, the content of which is incorporate herein by reference in its entirety). The amino acid sequence of GFP is provided as SEQ ID NO:2, and the amino acid sequence of NG is provided as SEQ ID NO:3. As such, suitable proteins for the fusion protein may include the amino acid sequence of SEQ ID NO:2 or SEQ ID NO:3 or variants thereof exhibiting at least about 50%, 60%, 70%, 80%, 90%, 95%, 96%, 97%, 98%, or 99% sequence identity, where preferably the protein or variant associates with of assembles in the biological structure.

Exemplary fusion proteins may include a fusion protein comprising the amino acid sequence of RSP3 (e.g., RSP3 of Chlamydomonas reinhardtii or a variant thereof) having fused at the C-terminus the amino acid sequence a fluorescent protein such as GFP, NG, or a variant thereof. In some embodiments, the fusion protein comprises the amino acid sequence of SEQ ID NO:4 or SEQ ID NO:5 or a variant thereof having at least about 50%, 60%, 70%, 80%, 90%, 95%, 96%, 97%, 98%, or 99% sequence identity to SEQ ID NO:4 or SEQ ID NO:5.

As indicated, the fusion proteins disclosed herein may comprise the amino acid sequence of a radial spoke protein (RSP) associated with a microtubule or a variant thereof fused to the amino acid sequence of a fluorescent protein or variant thereof. In some embodiments, the amino acid sequence of the fluorescent protein is fused to the C-terminus of the amino acid sequence of the RSP. Suitable RSPs may include, but are not limited to radial spoke protein 3 (RSP3). Suitable fluorescent proteins may include but are not limited to GFP, EGFP, NG, EBFP, ECFP, Midoriishi-Cyan1 protein, AmCyan1 protein, Azami-Green protein, mAzami-Green1 protein, ZsGreen1, EYFP, Venus protein, ZsYellow protein, Kusabira-Orange1 protein, and mKusabira-Orange1 protein.

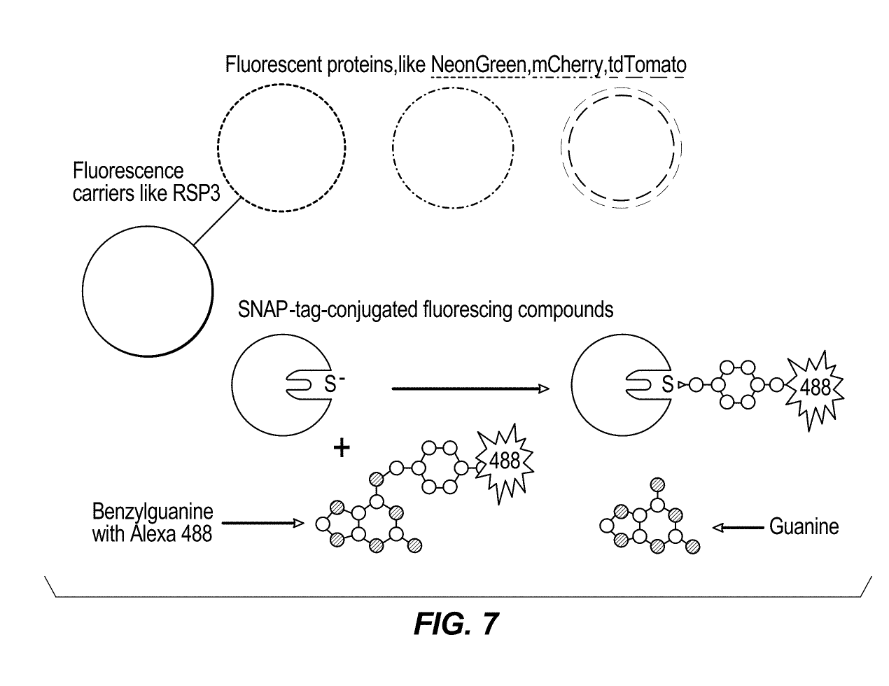

In some embodiments, the disclosed fusion proteins may comprise the amino acid sequence of a radial spoke protein (RSP) associated with a microtubule or a variant thereof fused to the amino acid sequence of an adapter protein (or a portion of an adapter protein (i.e., “an adapter portion”)) for binding a fluorophore as a fluorescent label, which may include a non-protein fluorophore rather than a fluorescent protein. For example, the disclosed fusion proteins may comprise an anchor portion fused to an adapter portion where the adapter portion of the fusion protein binds to a fluorophore label. Suitable adapter proteins may include biotinylated polypeptides that bind to streptavidin-conjugated fluorophore label, which may include non-protein fluorophore labels. As such, the disclosed fluorescently-labelled fusion proteins may comprise an anchor portion fused to biotinylated adapter polypeptide which binds to a streptavidin-conjugated fluorophore label. Examples of adapter proteins may include, but are not limited to biotinylated polypeptides such as AviTag or biotin carboxyl carrier protein (BCCP). and SNAP tag (available from New England BioLabs). The 15-a.a. AviTag, or 9-kD BCCP portion of a fusion protein can be biotinylated in vivo or in vitro using a BirA enzyme. The purified fusion protein or a purified biological structure comprising the fusion protein (e.g., biotinylated flagella) can be incubated with Streptavidin-conjugated fluorescing compounds in vitro. The SNAP-tag is a 20 kDa mutant of the DNA repair protein O⁶-alkylguanine-DNA alkyltransferase that reacts specifically and rapidly with benzylguanine (BG) derivatives of fluorophores leading to irreversible covalent labeling of the SNAP-tag with the BG-fluorophore. (See FIG. 7).

As such, in some embodiments of the fusion protein, the fluorescent protein portion is replaced by an adapter protein portion that binds a fluorophore, either covalently or non-covalently, as a fluorescent label. Suitable fluorophores may include but are not limited to 1,5 IAEDANS; 1,8-ANS; 4-Methylumbelliferone; 5-carboxy-2,7-dichlorofluorescein; 5-Carboxyfluorescein (5-FAM); 5-Carboxytetramethylrhodamine (5-TAMRA); 5-FAM (5-Carboxyfluorescein); 5-HAT (Hydroxy Tryptamine); 5-Hydroxy Tryptamine (HAT); 5-ROX (carboxy-X-rhodamine); 5-TAMRA (5-Carboxytetramethylrhodamine); 6-Carboxyrhodamine 6G; 6-CR 6G; 6-JOE; 7-Amino-4-methylcoumarin; 7-Aminoactinomycin D (7-AAD); 7-Hydroxy-4-methylcoumarin; 9-Amino-6-chloro-2-methoxyacridine; ABQ; Acid Fuchsin; ACMA (9-Amino-6-chloro-2-methoxyacridine); Acridine Orange; Acridine Red; Acridine Yellow; Acriflavin; Acriflavin Feulgen SITSA; Alexa Fluor 350™; Alexa Fluor 430™; Alexa Fluor 488™; Alexa Fluor 532™; Alexa Fluor 546™; Alexa Fluor 568™; Alexa Fluor 594™; Alexa Fluor 633™; Alexa Fluor 647™; Alexa Fluor 660™; Alexa Fluor 680™; Alizarin Complexon; Alizarin Red; Allophycocyanin (APC); AMC; AMCA-S; AMCA (Aminomethylcoumarin); AMCA-X; Aminoactinomycin D; Aminocoumarin; Aminomethylcoumarin (AMCA); Anilin Blue; Anthrocyl stearate; APC (Allophycocyanin); APC-Cy7; APTS; Astrazon Brilliant Red 4G; Astrazon Orange R; Astrazon Red 6B; Astrazon Yellow 7 GLL; Atabrine; ATTO-TAG™ CBQCA; ATTO-TAG™ FQ; Auramine; Aurophosphine G; Aurophosphine; BAO 9 (Bisaminophenyloxadiazole); Berberine Sulphate; Beta Lactamase; Bimane; Bisbenzamide; Bisbenzimide (Hoechst); Blancophor FFG; Blancophor SV; BOBO™-1; BOBO™-3; Bodipy 492/515; Bodipy 493/503; Bodipy 500/510; Bodipy 505/515; Bodipy 530/550; Bodipy 542/563; Bodipy 558/568; Bodipy 564/570; Bodipy 576/589; Bodipy 581/591; Bodipy 630/650-X; Bodipy 650/665-X; Bodipy 665/676; Bodipy FL; Bodipy FL ATP; Bodipy Fl-Ceramide; Bodipy R6G SE; Bodipy TMR; Bodipy TMR-X conjugate; Bodipy TMR-X, SE; Bodipy TR; Bodipy TR ATP; Bodipy TR-X SE; BO-PRO™-1; BO-PRO™-3; Brilliant Sulphoflavin FF; Calcein; Calcein Blue; Calcium Crimson™; Calcium Green; Calcium Orange; Calcofluor White; Carboxy-X-rhodamine (5-ROX); Cascade Blue™; Cascade Yellow; Catecholamine; CCF2 (GeneBlazer); CFDA; Chlorophyll; Chromomycin A; CL-NERF (Ratio Dye, pH); CMFDA; Coelenterazine f; Coelenterazine fcp; Coelenterazine h; Coelenterazine hcp; Coelenterazine ip; Coelenterazine n; Coelenterazine O; Coumarin Phalloidin; C-phycocyanine; CPM Methylcoumarin; CTC; CTC Formazan; Cy2™; Cy3.1 8; Cy3.5™; Cy3™; Cy5.1 8; Cy5.5™; Cy5™; Cy7™; cyclic AMP Fluorosensor (FiCRhR); Dabcyl; Dansyl; Dansyl Amine; Dansyl Cadaverine; Dansyl Chloride; Dansyl DHPE; Dansyl fluoride; DAPI; Dapoxyl; Dapoxyl 2; Dapoxyl 3; DCFDA; DCFH (Dichlorodihydrofluorescein Diacetate); DDAO; DHR (Dihydorhodamine 123); Di-4-ANEPPS; Di-8-ANEPPS (non-ratio); DiA (4-Di-16-ASP); Dichlorodihydrofluorescein Diacetate (DCFH); DiD—Lipophilic Tracer; DiD (DiIC18(5)); DIDS; Dihydorhodamine 123 (DHR); DiI (DiIC18(3)); Dinitrophenol; DiO (DiOC18(3)); DiR; DiR (DiIC18(7)); DNP; Dopamine; DsRed; DTAF; DY-630-NHS; DY-635-NHS; ELF 97; Eosin; Erythrosin; Erythrosin ITC; Ethidium Bromide; Ethidium homodimer-1 (EthD-1); Euchrysin; EukoLight; Europium (III) chloride; Fast Blue; FDA; Feulgen (Pararosaniline); FITC; Flazo Orange; Fluo-3; Fluo-4; Fluorescein (FITC); Fluorescein Diacetate; Fluoro-Emerald; FluoroGold (Hydroxystilbamidine); Fluor-Ruby; FluorX; FM 1-43™; FM 4-46; Fura Red™; Fura Red™/Fluo-3; Fura-2; Fura-2/BCECF; Genacryl Brilliant Red B; Genacryl Brilliant Yellow 10GF; Genacryl Pink 3G; Genacryl Yellow 5GF; GeneBlazer (CCF2); Gloxalic Acid; Granular Blue; Haematoporphyrin; Hoechst 33258; Hoechst 33342; Hoechst 34580; HPTS; Hydroxycoumarin; Hydroxystilbamidine (FluoroGold); Hydroxytryptamine; Indo-1; Indodicarbocyanine (DiD); Indotricarbocyanine (DiR); Intrawhite Cf; JC-1; JO-JO-1; JO-PRO-1; Laurodan; LDS 751 (DNA); LDS 751 (RNA); Leucophor PAF; Leucophor SF; Leucophor WS; Lissamine Rhodamine; Lissamine Rhodamine B; Calcein/Ethidium homodimer; LOLO-1; LO-PRO-1; Lucifer Yellow; Lyso Tracker Blue; Lyso Tracker Blue-White; Lyso Tracker Green; Lyso Tracker Red; Lyso Tracker Yellow; LysoSensor Blue; LysoSensor Green; LysoSensor Yellow/Blue; Mag Green; Magdala Red (Phloxin B); Mag-Fura Red; Mag-Fura-2; Mag-Fura-5; Mag-Indo-1; Magnesium Green; Magnesium Orange; Malachite Green; Marina Blue; Maxilon Brilliant Flavin 10 GFF; Maxilon Brilliant Flavin 8 GFF; Merocyanin; Methoxycoumarin; Mitotracker Green FM; Mitotracker Orange; Mitotracker Red; Mitramycin; Monobromobimane; Monobromobimane (mBBr-GSH); Monochlorobimane; MPS (Methyl Green Pyronine Stilbene); NBD; NBD Amine; Nile Red; Nitrobenzoxadidole; Noradrenaline; Nuclear Fast Red; Nuclear Yellow; Nylosan Brilliant Iavin E8G; Oregon Green; Oregon Green 488-X; Oregon Green™; Oregon Green™ 488; Oregon Green™ 500; Oregon Green™ 514; Pacific Blue; Pararosaniline (Feulgen); PBFI; PE-Cy5; PE-Cy7; PerCP; PerCP-Cy5.5; PE-TexasRed [Red 613]; Phloxin B (Magdala Red); Phorwite AR; Phorwite BKL; Phorwite Rev; Phorwite RPA; Phosphine 3R; Phycoerythrin B [PE]; Phycoerythrin R [PE]; PKH26 (Sigma); PKH67; PMIA; Pontochrome Blue Black; POPO-1; POPO-3; PO-PRO-1; PO-PRO-3; Primuline; Procion Yellow; Propidium lodid (PI); PyMPO; Pyrene; Pyronine; Pyronine B; Pyrozal Brilliant Flavin 7GF; QSY 7; Quinacrine Mustard; Red 613 [PE-TexasRed]; Resorufin; RH 414; Rhod-2; Rhodamine; Rhodamine 110; Rhodamine 123; Rhodamine 5 GLD; Rhodamine 6G; Rhodamine B; Rhodamine B 200; Rhodamine B extra; Rhodamine BB; Rhodamine BG; Rhodamine Green; Rhodamine Phallicidine; Rhodamine Phalloidine; Rhodamine Red; Rhodamine WT; Rose Bengal; R-phycocyanine; R-phycoerythrin (PE); SBFI; Serotonin; Sevron Brilliant Red 2B; Sevron Brilliant Red 4G; Sevron Brilliant Red B; Sevron Orange; Sevron Yellow L; SITS (Stilbene Isothiosulphonic Acid); SNAFL calcein; SNAFL-1; SNAFL-2; SNARF calcein; SNARF1; Sodium Green; SpectrumAqua; SpectrumGreen; SpectrumOrange; Spectrum Red; SPQ (6-methoxy-N-(3-sulfopropyl)quinolinium); Stilbene; Sulphorhodamine B can C; Sulphorhodamine G Extra; SYTO 11; SYTO 12; SYTO 13; SYTO 14; SYTO 15; SYTO 16; SYTO 17; SYTO 18; SYTO 20; SYTO 21; SYTO 22; SYTO 23; SYTO 24; SYTO 25; SYTO 40; SYTO 41; SYTO 42; SYTO 43; SYTO 44; SYTO 45; SYTO 59; SYTO 60; SYTO 61; SYTO 62; SYTO 63; SYTO 64; SYTO 80; SYTO 81; SYTO 82; SYTO 83; SYTO 84; SYTO 85; SYTOX Blue; SYTOX Green; SYTOX Orange; Tetracycline; Tetramethylrhodamine (TRITC); Texas Red™; Texas Red-X™ conjugate; Thiadicarbocyanine (DiSC3); Thiazine Red R; Thiazole Orange; Thioflavin 5; Thioflavin S; Thioflavin TCN; Thiolyte; Thiozole Orange; Tinopol CBS (Calcofluor White); TMR; TO-PRO-1; TO-PRO-3; TO-PRO-5; TOTO-1; TOTO-3; TriColor (PE-Cy5); TRITC TetramethylRodaminelsoThioCyanate; True Blue; TruRed; Ultralite; Uranine B; Uvitex SFC; WW 781; X-Rhodamine; XRITC; Xylene Orange; Y66F; Y66H; Y66W; YO-PRO-1; YO-PRO-3; YOYO-1; and YOYO-3.

Also contemplated herein are polynucleotides encoding the amino acid sequence of the fusion proteins disclosed herein. The polynucleotides may be operably linked to a promoter, for example within an expression vector.

Also contemplated are isolated cells comprising expression vectors that express the fusion proteins, for example, transformed or transfected cells. The isolated cells may be cultured in order to produce the fusion proteins and/or biological structures comprising the fusion proteins, such as microtubules, axonemes, and/or flagellum, for example where the fluorescent markers disclosed herein comprise the biological structures. Methods for preparing and isolating and/or purifying flagellum are known in the art. (See Craige et al., “Isolation of Chlamydomonas Flagella,” Curr. Prot. Cell Biol. 2013 June; 0 3: Unit-3.41.9, the content of which is incorporated herein by reference in its entirety). Isolated fluorescent flagellum prepared as disclosed herein may be further processed to isolated the axoneme and/or microtubules.

The disclosed fluorescent markers optionally may be immobilized on a solid substrate, for example, a microscopic slide. The microscopic slide having the fluorescent marker immobilized thereon may be utilized in fluorescence microscopy for analyzing and imaging a fluorescently-labeled sample.

Also contemplated herein are methods for performing fluorescence microscopy. The methods utilize the fluorescent markers disclosed herein and may include a step of detecting fluorescence from the fluorescent marker or from a solid substrate having the fluorescent marker immobilized thereon while performing fluorescence microscopy and/or imaging the fluorescent marker. In some embodiments of the methods, the fluorescent marker may be applied to a solid substrate such as a microscopic slide, and subsequently a fluorescently-labeled sample is applied to the slide prior to performing fluorescence microscopy. Fluorescence then is detected from the fluorescent marker and/or the fluorescent marker is imaged, and then, either concurrently or non-concurrently, fluorescence is detected from the fluorescently-labeled sample and/or the fluorescently-labeled sample is imaged, while performing fluorescence microscopy.

Alternatively, the fluorescent marker may be pre-provided on a solid substrate such as a microscopic slide where the fluorescent marker is immobilized thereon, such that a user need not apply the fluorescent marker to the solid substrate, and the user then applies the fluorescently-labeled sample to the solid substrate. Fluorescence then is detected from the fluorescent marker and/or the fluorescent marker is imaged, and then, either concurrently or non-concurrently, fluorescence is detected from the fluorescently-labeled sample and/or the fluorescently-labeled sample is imaged, while performing fluorescence microscopy.

In the methods for performing fluorescence microscopy, the fluorescent label of the marker may be the same as or different than the fluorescent label of the sample. The fluorescent marker may be imaged separated or together with the fluorescently-labeled sample.

### ILLUSTRATIVE EMBODIMENTS

The following embodiments are illustrative and should not be interpreted to limit the scope of the claimed subject matter.

### Embodiment 1

A fluorescent marker comprising a tubular or cylindrical biological structure formed by multiple copies of a structural protein (SP), the biological structure comprising multiple copies of a fluorescently-labeled protein (FP) regularly interspersed along the length of the biological structure.

### Embodiment 2

The fluorescent marker of embodiment 1 comprising two FPs per 96 nm length of the biological structure.

### Embodiment 3

The fluorescent marker of embodiment 1 or 2, wherein the structural proteins are assembled in a helical configuration.

### Embodiment 4

The fluorescent marker of any of the foregoing embodiments, wherein the structural protein comprises tubulin.

### Embodiment 5

The fluorescent marker of embodiment 4, wherein the tubulin is α-tubulin (SEQ ID NO:6 or a variant thereof), β-tubulin (SEQ ID NO:7 or a variant thereof), or a combination of α-tubulin and β-tubulin as a heterodimer.

### Embodiment 6

The fluorescent marker of any of the foregoing embodiments, wherein the biological structure is a microtubule or a doublet microtubule.

### Embodiment 7

The fluorescent marker of any of the foregoing embodiments, wherein the biological structure is a proteinaceous microtubule or a proteinaceous doublet microtubule.

### Embodiment 8

The fluorescent marker of any of the foregoing embodiments, wherein the biological structure is a doublet microtubule comprising an A-microtubule and a B-microtubule.

### Embodiment 9

The fluorescent marker of any of the foregoing embodiments, wherein the fluorescently-labeled protein is a fusion protein comprising the amino acid sequence of a radial spoke protein (RSP) associated with a microtubule fused to the amino acid sequence of a fluorescent protein.

### Embodiment 10

The fluorescent marker of embodiment 9, wherein the amino acid sequence of the fluorescent protein is fused to the C-terminus of the amino acid sequence of the RSP.

### Embodiment 11

The fluorescent marker of embodiment 9 or 10, wherein the fluorescent protein is green fluorescent protein (GFP), mNeonGreen protein (NG), or a fluorescent variant thereof.

### Embodiment 12

The fluorescent marker of any of embodiments 9-11, wherein the RSP is radial spoke protein 3 (RSP3) or a variant thereof that assembles into a microtubule structure.

### Embodiment 13

The fluorescent marker of embodiment 12, wherein the RSP3 comprises the amino acid sequence of SEQ ID NO: 1 or a variant thereof having at least about 50%, 60%, 70%, 80%, 90%, 95%, 96%, 97%, 98%, or 99% sequence identity to SEQ ID NO:1.

### Embodiment 14

The fluorescent marker of any of embodiments 9-13, wherein the fluorescent protein comprises the amino acid sequence of SEQ ID NO:2 or SEQ ID NO:3 or a variant thereof having at least about 50%, 60%, 70%, 80%, 90%, 95%, 96%, 97%, 98%, or 99% sequence identity to SEQ ID NO:2 or SEQ ID NO:3.

### Embodiment 15

The fluorescent marker of any of embodiments 9-12, wherein the fusion protein comprises the amino acid sequence of SEQ ID NO:4 or SEQ ID NO:5 or a variant thereof having at least about 50%, 60%, 70%, 80%, 90%, 95%, 96%, 97%, 98%, or 99% sequence identity to SEQ ID NO:4 or SEQ ID NO:5.

### Embodiment 16

The fluorescent marker of any of the foregoing embodiments, wherein the fluorescently-labeled protein is a fusion protein comprising the amino acid sequence of a radial spoke protein (RSP) fused to the amino acid sequence an adapter protein that binds to a fluorescent label.

### Embodiment 17

The fluorescent marker of any of the foregoing embodiments, wherein the biological structure is an axoneme comprising multiple copies of the tubular or cylindrical biological structure.

### Embodiment 18

The fluorescent marker of embodiment 17, wherein the biological structure is an axoneme comprising a 9+2 structure.

### Embodiment 19

The fluorescent marker of any of the foregoing embodiments, wherein the biological structure is a flagellum comprising an axoneme

### Embodiment 20

The fluorescent marker of embodiment 19, wherein the axoneme comprises multiple copies of the tubular or cylindrical biological structure.

### Embodiment 21

A solid substrate comprising the fluorescent marker of any of the foregoing embodiments immobilized to the solid substrate.

### Embodiment 22

The solid substrate of embodiment 21, wherein the solid substrate is a slide.

### Embodiment 23

A method for performing fluorescence microscopy, the method comprising detecting fluorescence from the fluorescent marker of any of embodiments 1-20 or from the solid substrate of embodiment 21 or 22 comprising the fluorescent marker immobilized to the solid substrate while performing fluorescence microscopy.

### Embodiment 24

A method for performing fluorescence microscopy, the method comprising applying the fluorescent marker of any of embodiments 1-20 to a substrate, and detecting fluorescence from the fluorescent marker while performing fluorescence microscopy.

### Embodiment 25

The method of embodiment 24, wherein the solid substrate is a slide.

### Embodiment 26

A method of embodiment 24 or 25, further comprising applying a fluorescently sample to the same substrate or to a different substrate and detecting fluorescence from the sample while performing fluorescence microscopy.

### Embodiment 27

The method of embodiment 26, wherein the fluorescent label of the marker is the same as the fluorescent label of the sample.

### Embodiment 28

The method of embodiment 26, wherein the fluorescent label of the marker is different than the fluorescent label of the sample.

### Embodiment 29

A fusion protein comprising the amino acid sequence of a radial spoke protein (RSP) associated with a microtubule fused to the amino acid sequence of a fluorescent protein.

### Embodiment 30

The fusion protein of embodiment 29, wherein the amino acid sequence of the fluorescent protein is fused to the C-terminus of the amino acid sequence of the RSP.

### Embodiment 31

The fusion protein of embodiment 29 or 30, wherein the fluorescent protein is green fluorescent protein (GFP), mNeonGreen protein (NG), or a fluorescent variant thereof.

### Embodiment 32

The fusion protein of any of embodiments 29-31, wherein the RSP is radial spoke protein 3 (RSP3) or a variant thereof that assembles into a microtubule structure.

### Embodiment 33

The fusion protein of embodiment 32, wherein the RSP3 comprises the amino acid sequence of SEQ ID NO: 1 or a variant thereof having at least about 50%, 60%, 70%, 80%, 90%, 95%, 96%, 97%, 98%, or 99% sequence identity to SEQ ID NO:1.

### Embodiment 34

The fusion protein of any of embodiments 29-33, wherein the fluorescent protein comprises the amino acid sequence of SEQ ID NO:4 or SEQ ID NO:5 or a variant thereof having at least about 50%, 60%, 70%, 80%, 90%, 95%, 96%, 97%, 98%, or 99% sequence identity to SEQ ID NO:4 or SEQ ID NO:5.

### Embodiment 35

The fusion protein of any of embodiments 29-34, wherein the fusion protein comprises the amino acid sequence of SEQ ID NO:4 or SEQ ID NO:5.

### Embodiment 36

A fusion protein comprising the amino acid sequence of a radial spoke protein (RSP) fused to the amino acid sequence an adapter protein that binds to a fluorescent label.

### Embodiment 37

A polynucleotide encoding the amino acid sequence of the fusion protein of any of embodiments 29-36.

### Embodiment 38

An expression vector comprising the polynucleotide of embodiment 37 operably linked to a promoter.

### Embodiment 39

An isolated cell comprising the expression vector of embodiment 38.

### Embodiment 40

The isolated cell of embodiment 39, wherein the isolated cell is a prokaryotic cell.

### EXAMPLES

The following Examples are illustrative only and do not limit the scope of the claimed subject matter.

Flagella Markers for Fluorescent Microscopy

Abstract

Despite surging interests in quantitative analysis, it remains cumbersome to estimate the numbers of molecules using fluorescent microscopy. Here we report the creation of biological fluorescent intensity standards, equivalent to protein or DNA markers for electrophoresis. We took advantage of a well-defined protein, RSP3 in the flagella of Chlamydomonas and a new fluorescent protein, mNeonGreen (NG) that has a similar excitation and emission spectra as enhanced green fluorescent protein (EGFP) but is brighter and switchable. RSP3 is a homodimer in the radial spoke complex that is assembled into each of the 9 microtubule doublets in the axoneme at an exact 32-64 nm periodicity. With on average 36 NG molecules per 96 nm, the flagella of Chlamydomonas RSP3-NG transgenic strains glow evenly. The intensity reduced 60% following methanol fixation that also favorably diminished chlorophyll-derived autofluorescence. The intensity nearly doubled at overlapped regions and was prorated at splayed doublets and fragmented doublet particles. The utility as an intensity standard was demonstrated by the comparison of RSP3-NG flagella with a NG fusion protein in Chlamydomonas, and with GFP fusion proteins in Saccharomyces cerevisiae. Fluorescent flagella, with different fluorescent tags and axonemal carrier proteins, will simplify quantitative fluorescence imaging.

Introduction

Fluorescence microscopy for biomedical sciences has come a long way. The fluorophores have expanded to a wide array of chemical compounds and fluorescent proteins that have distinct spectral properties suitability for various applications. With various ingenious tools, digital software and invention of new microscopes and image processing, it is now possible to use fluorescence in diagnosis and research that were in conceivable a decade prior.

Despite the tremendous progress, it remains cumbersome to deduce the number of molecules simply based on the intensity of fluorescent images. Fluorescent intensity is affected by excitation light intensity and spectra, which in turn are influenced by the age and condition of the lamps. Furthermore, intense excitation light will saturate fluorophores and, importantly, could bleach fluorophores. The brightness, contrast and the linear range could be further manipulated by gain during image acquisition and by the subsequent image processing that is designed to maximize the sensitivity and image quality. As such quantitative analysis of fluorescent images was often expressed in relative terms or requires elaborate instrumentation and calculations.

One solution is to include a standard, ideally during imaging, akin to a protein marker or a DNA ladder widely used in electrophoresis that reveals the sizes of unknown molecules based on their migration distances and their abundance based on their intensity after staining with dyes. Although there are various fluorescent standards ranging from quantum dots to DNA-origami for different applications (Michalet et al., 2005; Schmied et al., 2012, 2014), we reason that motile flagella of eukaryotic cells could be readily converted into fluorescent standards of appropriate intensity, scale and biocompatibility to be included in imaging of average biological samples.

Motile flagella, or the synonymous cilia, beat rhythmically to sweep surrounding fluid. Diverse eukaryotic cells use the movement for critical actions such as mating, food gathering and avoidance of noxious environments. In spite of the distinct movement, most of cilia and flagella are powered by a 9+2 axoneme with 9 microtubule doublets encircling two singlet microtubules in the center. Both types of microtubules associate with a variety of molecular complexes with distinctive functions. The best characterized are axonemal dyneins and radial spokes that anchor to each 9 outer doublet at a precise location periodically throughout the length of flagella. Dynein motors project toward neighboring doublets to drive inter-doublet sliding that is converted into rhythmic beating. Radial spokes direct toward the central pair to further control the dynein-driven motility. These complexes engage neighboring structures repetitively enabling the axoneme to beat rhythmically at high frequencies as a nanomachine. We took advantage the biflagellate algae, Chlamydomonas to generate flagella with fluorescent axonemes in which fluorescent proteins were carried by a well-defined radial spoke protein of a known stoichiometry. As such fluorescent proteins of a known quantity were distributed at precise locations of similar abundance throughout the length of flagella. We further used three examples to demonstrate their utility as intensity standards for quantitative analysis of fluorescent images.

Results

Generation of Fluorescent Flagella with RSP3 as a Fluorophore Carrier.

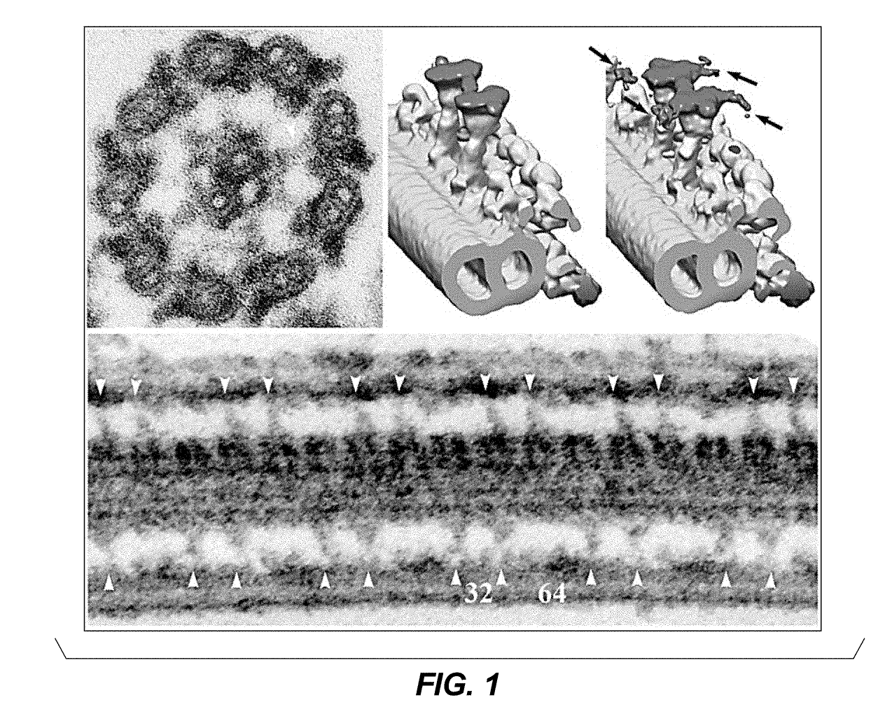

Aside from tubulins, axonemes are comprised of more than 400 distinct proteins in a number of molecular complexes that vary in periodicities within the 96 nm repeat. In theory, many axonemal proteins could carry a fluorophore without affecting their function. For this proof-of-principle study, we used the well characterized RSP3 in the radial spoke (RS) complex as the fluorescent protein carrier because of known stoichiometry of the RS and RSP3. Under electron microscopy (EM), the RS appears like a Y-shaped complex anchoring to outer doublets with its stalk, while projects its enlarged head toward the central pair apparatus (Huang et al., 1981; Pigino et al., 2011; FIG. 1A). Physical interactions of RSs and the central pair coordinate the activation of dynein motors on the outer doublets and are critical for the rhythmic beating. In Chlamydomonas, two RSs are positioned 32 nm apart every 96 nm along the length of each outer doublet (FIG. 1B). This corresponds to 18 RSs for every 96 nm flagella that have 9 outer doublets.

RSP3 exists as a homodimer spanning the RS as a scaffold for docking the other radial spoke subunits (Wirschell et al., 2008; Sivadas, et al., 2012; Oda et al., 2014). Chlamydomonas RSP3 mutant, pf14, generates paralyzed flagella that lack RSs (Huang et al., 1981; Diener et al., 1993). The deficiencies could be restored by transforming RSP3 genomic DNA—original, or with a tag (Williams et al., 1989; Gupta et al., 2012; Oda et al., 2014). Analysis of RSP3 deletion mutants and cryo-electron tomography of tagged RSP3 strains (FIG. 1C) positioned the C-terminal tail toward the spokehead (Sivadas et al., 2012; Oda et al., 2014). Therefore, the flagella of transgenic pf14 strains with fully rescued RSP3 tagged with a fluorescent protein (FP) should contain 36 RSP3, and thus 36 FPs every 96 nm.

Toward this end, we created RSP3 genomic constructs, inserting a DNA fragment encoding enhanced Green Fluorescent Protein (GFP) or mNeonGreen (NG) before the stop codon. GFP was tagged to a couple of axonemal proteins (Bower et al., 2013; Yanagisawa et al., 2014) and NG was tagged to EB1, a protein that bind preferentially to the tip of growing microtubules and enriched at the tip of flagella (Pederson et al., 2003; Harris et al., 2015). NG was chosen for three reasons. It is bright—2.7× fold brighter than EGFP, resulted from a 2.07× higher extinction coefficient and a 1.33× fold higher quantum yield (Shaner et al., 2013). In fact, its brightness is comparable to, if not higher than, most of the existing FPs. In addition, its photostability is comparable to GFP under widefield illumination. Lastly, NG has a similar excitation and emission spectra as GFP.

Comparison of Flagella with RSP3-GFP and RSP3-NG.

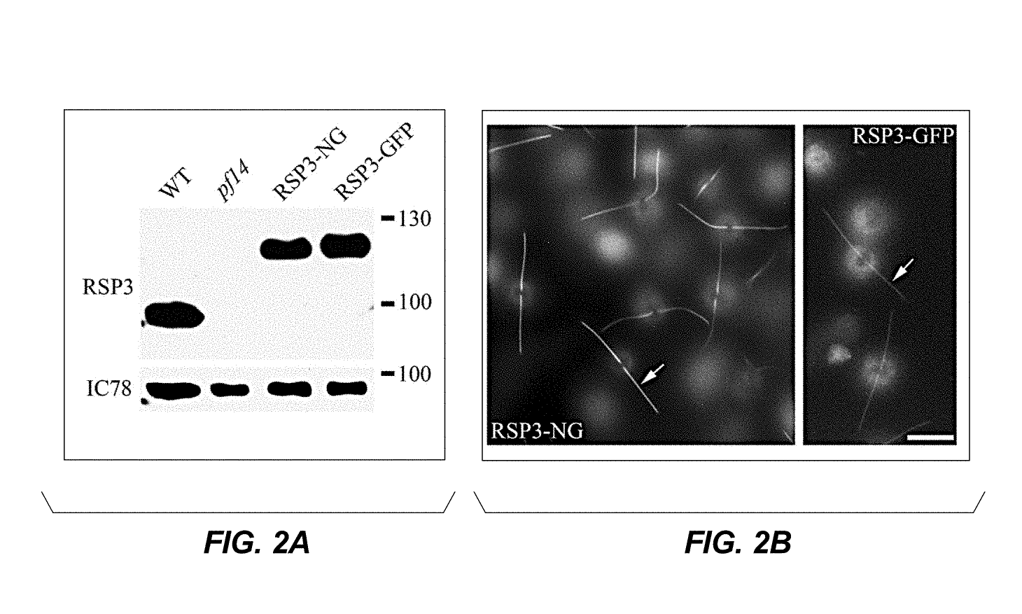

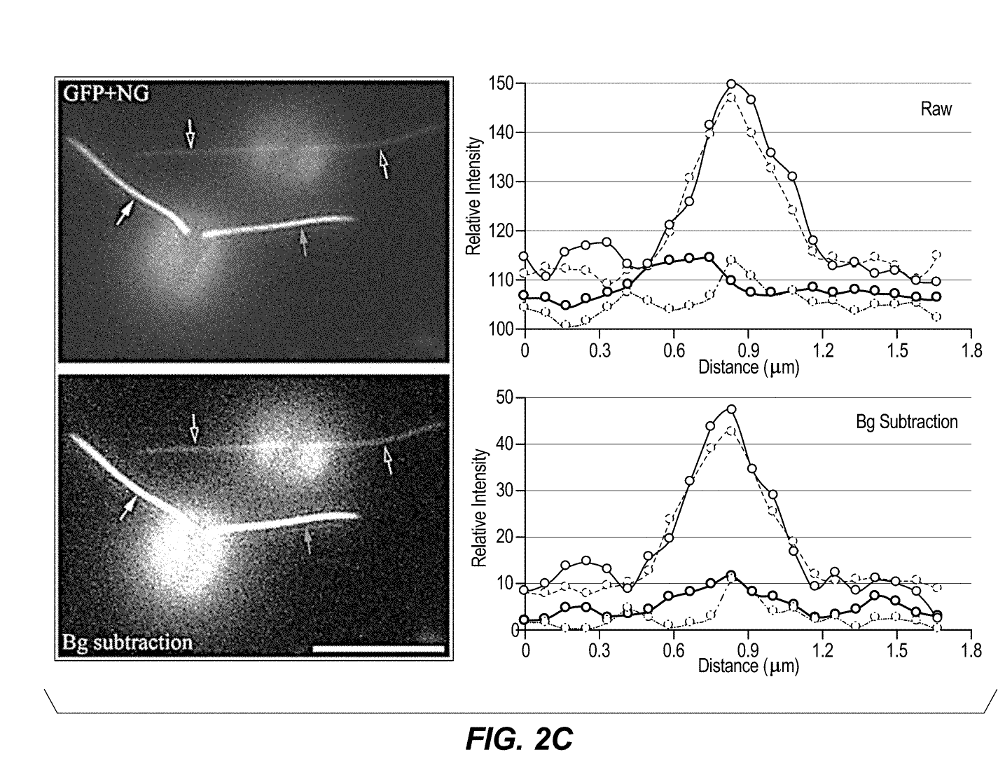

The plasmids that contained a RSP3-FP genomic construct and a paromomycin-resistant gene were transformed into the paralyzed RSP3 mutant pf14. Once introduced into cells, the plasmid would insert randomly into genome. A fraction of insertional events led to the expression of paromomycin-resistant gene and the genomic construct. Approximately 30% of antibiotic resistant clones were motile. The fully rescued clones—all cells swimming like wild types (WT)—were selected. Western blots of flagella showed that RSP3-GFP and RSP3-NG were similarly abundant as RSP3 in WT (FIG. 2A). The negative control was pf14. The loading control was IC78, a subunit in outer dynein arms that were normal in all four strains.

For fluorescence microscopy, we took advantage of algae's phototactic response. Algal cells would swim toward or away from the light source of the microscope and thus encountered the slide or coverslip. This led to flagella stuck to the glass surface and becoming quiescent, whereas the cell body of intense autofluorescence mostly from chlorophyll was out of focus. Quiescent flagella were imaged with a typical widefield epifluorescence microscope and a 40× objective lens. All images were acquired in a similar manner unless stated otherwise. Both RSP3-NG and RSP3-GFP appeared evenly distributing throughout entire flagella except the end where outer doublets taper off (FIG. 2B). When imaged together, it became evident that RSP3-GFP flagella were dimmer, barely distinguishable from the autofluorescence with a 1-sec exposure (FIG. 2C, upper panel). They became more evident after background subtraction (lower panel). ImageJ Plot Profile was used to measure fluorescent intensity across flagella (FIG. 2C, right panels). The peak values showed that RSP3-NG flagella were approximately 4× brighter than RSP3-GFP ones. The remaining study focused on RSP3-NG flagella, since their intensity level is closer to most studies using EGFP.

Characterization of RSP3-NG Flagella.

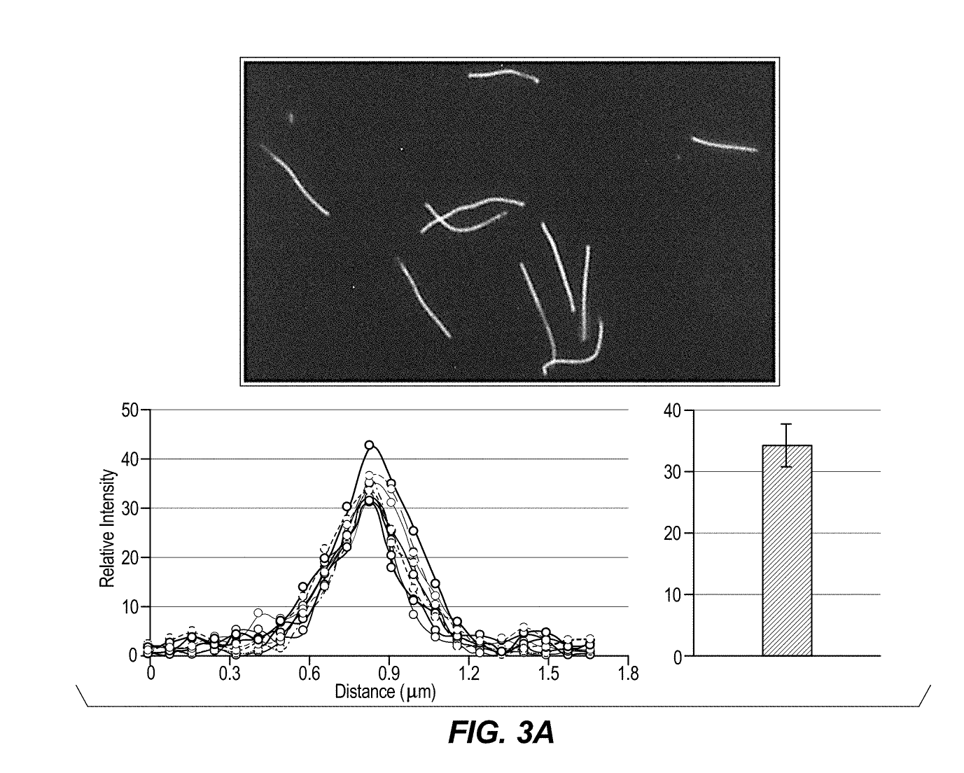

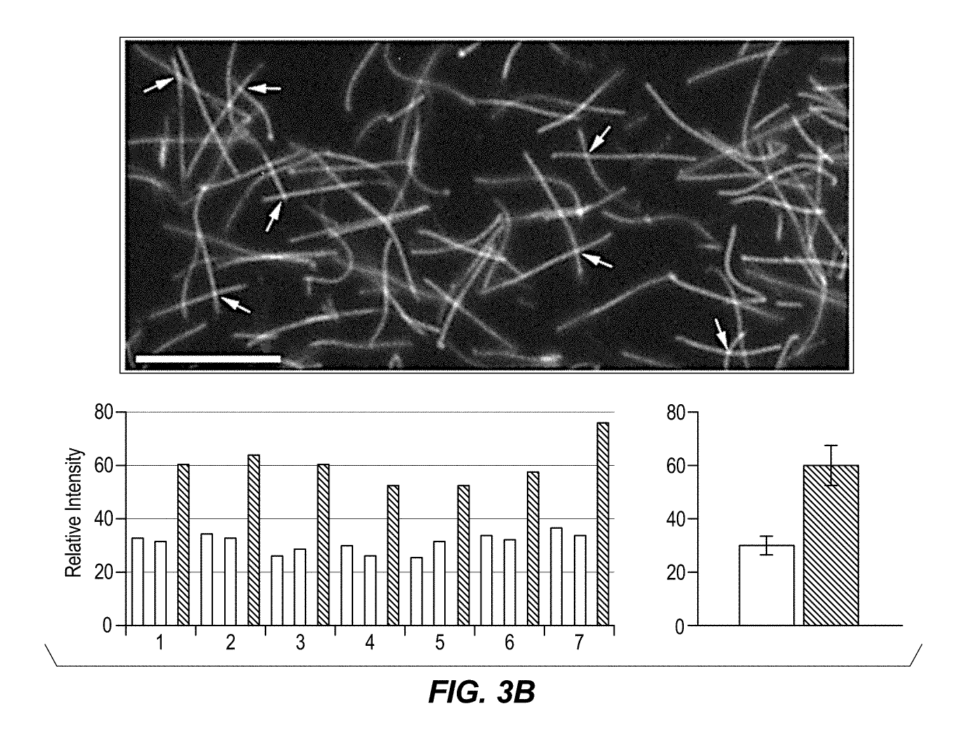

RSP3-NG flagella were excised from the transgenic cells for further characterizations. Most isolated flagella were 10-12 μm, some varying in fluorescent intensity due to uneven focal planes (FIG. 3A, top panel). A profile plot was made along each flagellum to determine the brightest region that was in focus. The profile plots across the brightest region for all flagella were compiled together (bottom panel). The average and standard deviation of the peak values were shown in a histogram (right panel).

To test if the intensity level of RSP3-NG molecules was linear to the molecule number, we compared the intensity of the overlapped region of two flagella and the nearby non-overlapped region (FIG. 3B, top panel). The peak intensity of the two regions (bottom left panel) and the averages (bottom right panel) indicated that the intensity at the two-flagellum regions was about 2× brighter than the single-flagellum regions as expected.

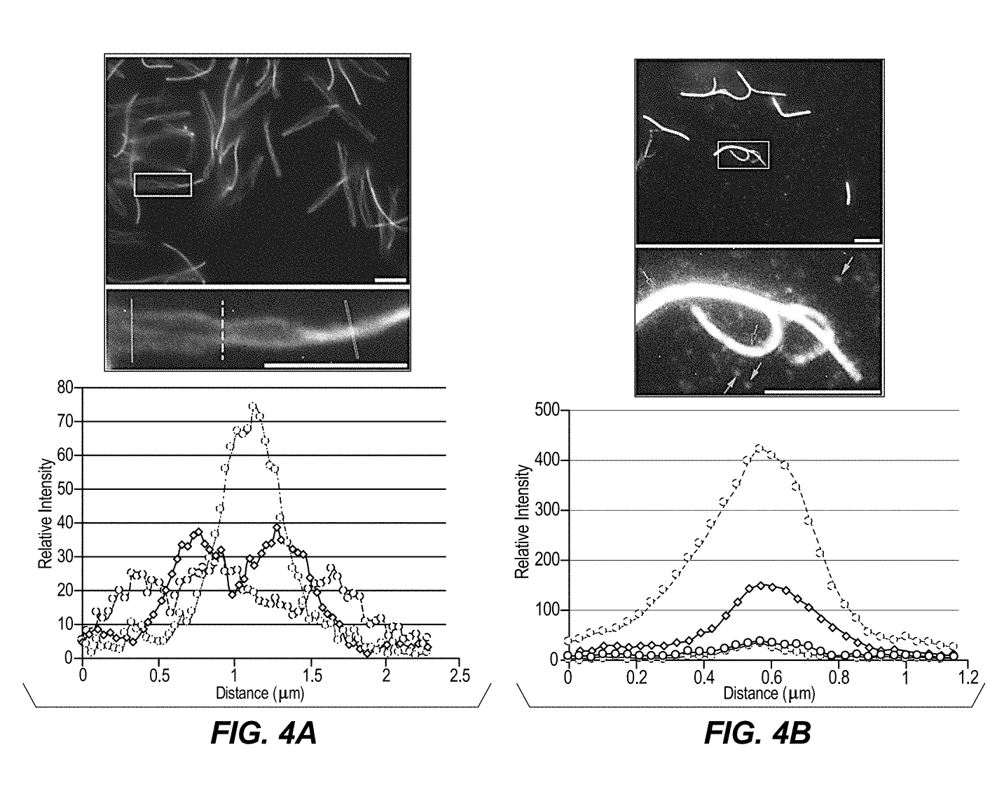

As RSP3-GFP flagella were dim but visible, we reasoned that split RSP3-NG outer doublet sub-fibers might be visible. Typically, outer doublets were splayed in the buffer containing a detergent for dissolving flagellar membrane, ATP consumed by dynein motors to power inter-doublet sliding, and a protease for cleaving mechanical constrains—including RSs. To maintain intact RSs, we applied shear force to flagella immobilized to poly-L-lysine by manually moving the coverslip back and forth. Under fluorescence microscopy, many flagella were splayed (FIG. 4A)—the 9 outer-doublet bundle split into 2 or more sub-fibers. Analysis of enlarged images taken with a 100× lens (FIG. 4A, middle panel) showed that fluorescence still evenly distributed along sub-fibers. Measurement of the fluorescence intensity showed that the more the sub-fibers were splayed, the lower the intensity of individual sub-fibers (bottom panel). The splaying was less extensive for unattached flagella (FIG. 4B, top panel). But under a 100× objective and with enhanced contrast, we found that some outer doublets were fragmented into particles (FIG. 4B, middle panel). The nearly one-ninth of intensity of individual particles relative to the intact region (FIG. 4B, bottom panel) suggested that they may consist of a single outer doublet, perhaps a 96-nm repeat. Therefore, flagella or outer doublets with RSP3-NG appear to be nearly proportional to the abundance of NG molecules.

NG Fluorescence after Methanol Fixation.

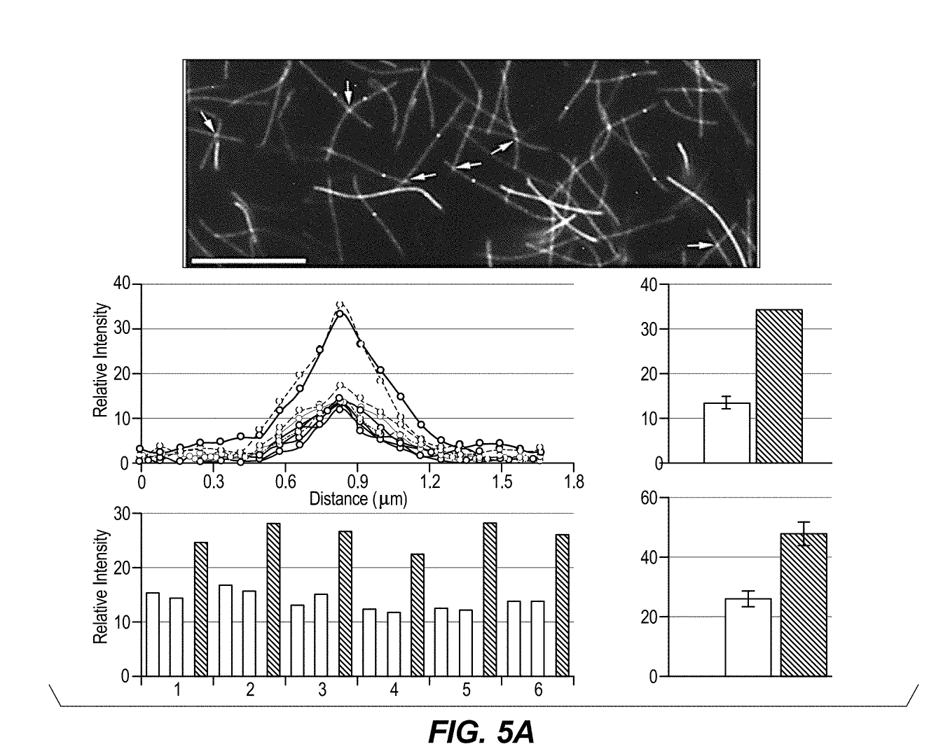

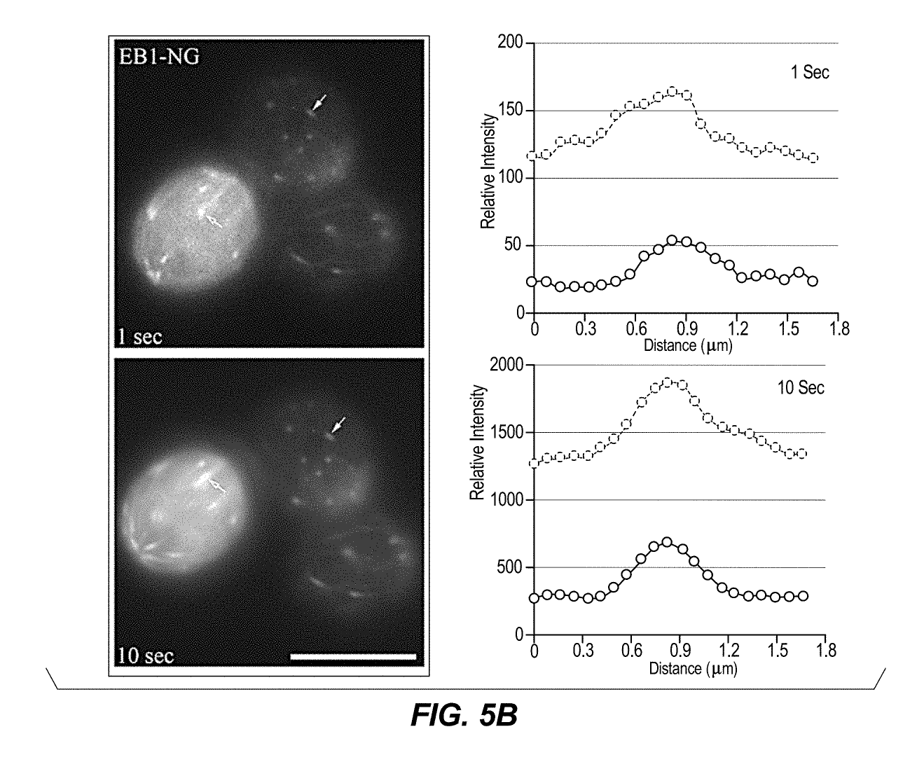

Although FPs are prized for live cell imaging, occasionally they need to be visualized in fixed cells. Rapid methanol fixation is commonly used to preserve dynamic microtubules in tissues and, for plants and Chlamydomonas, to extract pigments that contribute to the intense autofluorescence. However, GFP was often rendered invisible following methanol fixation and was instead visualized by immunofluorescence. To learn the effect of methanol on NG, slides with RSP3-NG flagella were submerged in −20° C. methanol for 20 mins. After dehydration and rehydration, fresh RSP3-NG flagella were added to the slide before imaging (FIG. 5A, top panel). The profile plot (middle left panel) of fixed flagella (red dots) and unfixed ones (blue dots) were analyzed. Averages of the peak values (middle right panel) indicated that methanol fixation reduced NG fluorescence intensity by ˜60%. Similarly, the overlapped regions (white arrows and grey bars) of fixed flagella were about twice as bright as non-overlapped regions (red bars).

We further tested a Chlamydomonas transgenic wild type strain expressing EB1-NG (Harris et al., 2016) with methanol fixation. The preferential plus end binding of growing microtubules rendered a typical comet pattern of fluorescent EB1. EB1-NG cells immobilized to poly-L-lysine coated slides were fixed in methanol first. After rehydration, live cells were added into the slide and images were acquired. While the intensity of EB1-NG comets in methanol-fixed cells (orange arrow in FIG. 5B, left panels) were dimmer than in live cells (blue arrow), autofluorescence also decreased substantially (FIG. 5B). Profile plots (right panels) showed that methanol treatment did not significantly affect the signal/background ratio. Comets were visible after a 10-second exposure (top panel) and remained visible after the subsequent 1-second exposure (bottom panel), showing NG's stability at this intensity of excitation light regardless of methanol fixation. As expected, the comet pattern appeared identical in fixed cells in the two images with different exposure, whereas the length of moving comet heads in live cells appeared to be ˜500 nm as reported (Seetapun et al., 2012) in the image of 1-sec exposure and longer in 10-sec image (blue arrow). Therefore, NG and RSP3-NG flagella are viable tools for experiments that involve methanol fixation.

Application of RSP3-NG Flagella to Different Molecules and Different Organisms.

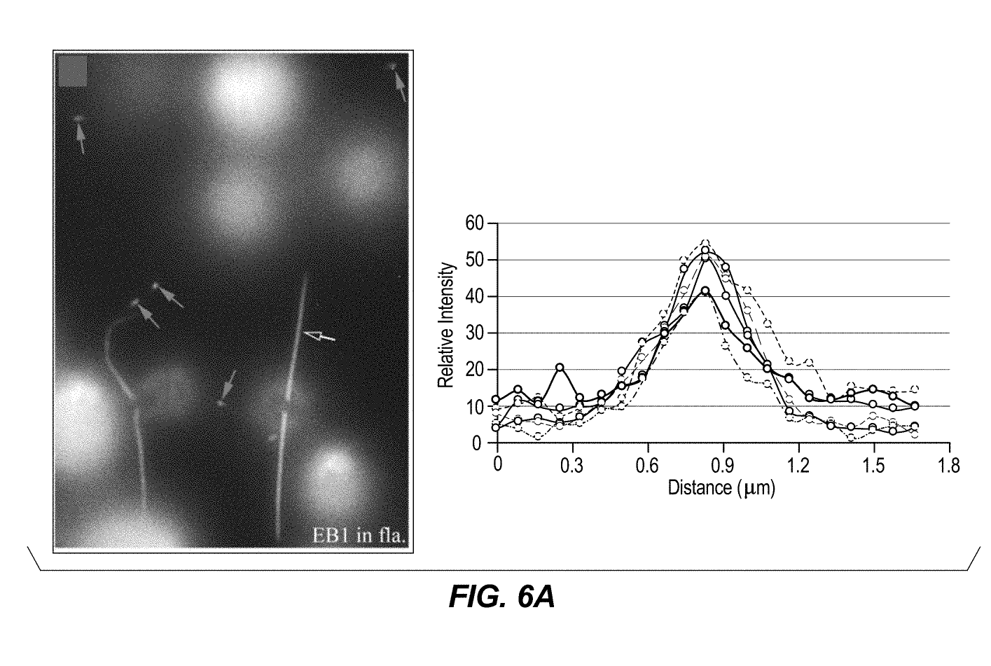

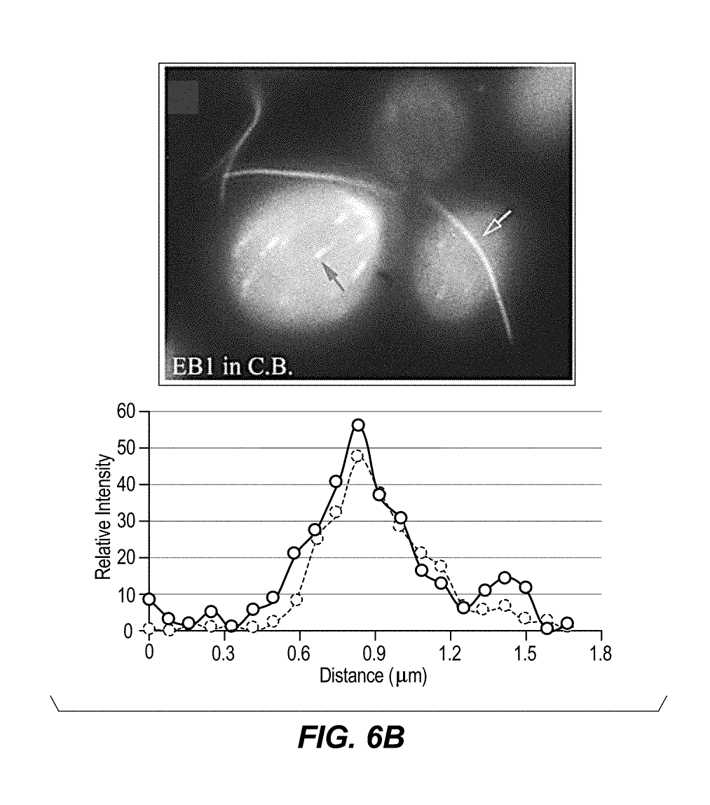

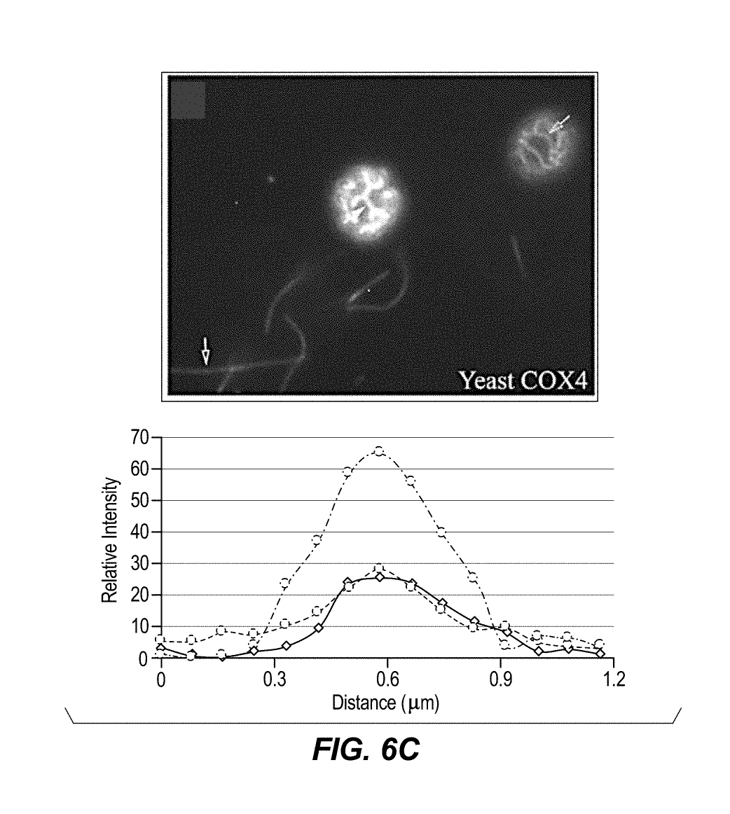

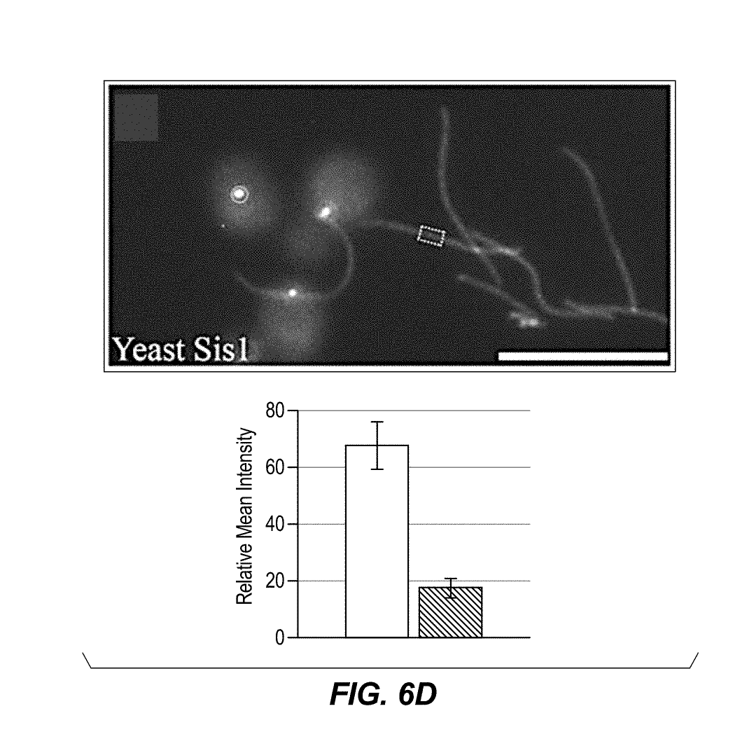

Given the linear relationship of RSP3-NG numbers and fluorescent intensity, we reason that it is feasible to deduce fluorescent molecule numbers regardless of the cellular compartments and cell types. We first compare RSP3-NG flagella with EB1-NG cells. Aside from EB1-NG comets in the cell body, EB1-NG is also enriched at the flagellar tip where microtubule plus ends undergo constant turn over even after flagella reach full lengths (Pederson et al., 2003; Harris et al., 2016; Marshall and Rosenbaum, 2001). Fluorescence Recovery after Photobleaching (FRAP) analysis suggests that the tip population contains both immobile EB1-NG and a highly dynamic population that may account for tubulin turnover at the plus end (Harris et al., 2016). To evaluate the quantity of EB1-NG at the flagellar tip, we image RSP3-NG cells and EB1-NG cells together, focusing flagella adhering to the glass surface at the same focal plane (FIG. 6A). EB1-NG tips were about 500 nm in length. Profile plots indicated that the intensity of EB1-NG tips was similar to, or dimmer than, that of RSP3-NG flagella. To analyze EB1-NG comets in the cell body, the cells were co-imaged with isolated flagella. Analysis of comets and flagella at a similar focal plane showed that the leading end of comet heads at individual microtubules' plus end (FIG. 6B, red arrow) was as bright as RSP3-NG flagella, if not brighter. We deduced that a 500-nm comet head, which corresponds to 5.2 96-nm repeats, each containing 36 RSP3-NG molecules, may contain roughly 187 EB1-NG, or 93 EB1-NG dimers recruited to the tip of a single growing microtubule. Given the presence of untagged EB1 of similar abundance (Harris et al., 2016), the brightest 500 nm comet head should have 187 EB1 dimers, including the tagged and untagged EB1, assuming C-terminal tagging does not substantially affect plus end tracking. This measurement in green algae at room temperature is in line with the 270 EB dimers for a 1-μm microtubule plus end in epithelial cells measured at 37° C. (Seetapun et al., 2012). The similar EB1-NG intensity at the tip of one microtubule and at the tip of a full-length flagellum that contain 20 microtubules from 9 outer doublet microtubules and 2 microtubule singlets showed that despite the constant tubulin-turnover at the tip of a full length flagella, the rate is ˜1/20 of that at the plus end of growing microtubules in the cell body.

We also compared RSP3-NG flagella with two transgenic Saccharomyces cerevisiae strains. One strain expressed GFP tagged to the first 21 amino acids in Cytochrome c oxidase subunit 4 (COX4) (Jensen et al., 2000). Cytochrome c complex associates with the matrix surface of the inner membrane in mitochondria. COX4-GFP that illuminates the mitochondrial reticular network in yeast is a powerful tool to screen for yeast mitochondria mutants differing in fission, fusion and shape. COX4-GFP level varied among individual yeast cells (Jensen et al., 2000). The peak intensity in a low abundance cell (FIG. 6C, right) was similar to that of RSP3-NG flagella. Given that GFP is 2.7× less bright than NG, every 96-nm long mitochondrial tubule is expected to contain ˜97 (2.7×36) COX4-GFP molecules. This estimate is slightly lower than actual numbers since the average diameter of a tubular mitochondrion is 300-400 nm, larger than the ˜220-nm diameter of axonemes (Westermann, 2008). The abundance of COX4-GFP is 2 fold higher in the cells to the left.

The other yeast strain expressed GFP-tagged Sis1, a HSP40 co-chaperon of HSP70. It is involved in the trafficking of misfolded polypeptides to the insoluble protein deposit (IPOD) compartment as a part of cellular strategies for controlling protein aggregates (Kaganovich et al., 2008; Specht et al., 2011; Nillegoda et al., 2015). Sis1-GFP molecules enriched in the non-membrane bound IPOD appeared as a puncta of a diameter larger than that of flagella (FIG. 6D). Based on the formula outlined in Material and Methods, each spot on average contains ˜2,000 Sis1-GFP molecules at the permissive temperature 30° C., approximately 1/10 of the 20,500 Sis1 molecules estimated in one yeast cell (Ghaemmaghami et al., 2003).

Expand the Variety of Flagellar Standards for Diverse Applications.

So far we only created fluorescent flagella with either RSP3-GFP or RSP3-NG and tested them with single cells. Both flagella emit green light albeit with different intensities. It is necessary to diversify flagellar standards to tap into the vast market that encompasses various applications utilizing a wide array of fluorescing tools with different properties, colors and applications. 2A) One is to switch NeonGreen to two major types of tags (FIG. 3). The existing DNA constructs are designed for easy swaps of tags. One is fluorescent proteins of other colors, such as mCherry or tdTomato that are at the red light range. Since fluorescent proteins must be expressed by cells and thus are usually used in live cell imaging. The other option is the versatile SNAP-tag protein which by itself does not emit light but could be linked via chemical reactions to commercially available compounds that emit light as illustrated in FIG. 3. Contrary to fluorescent proteins that are limited to live cells, fluorescing compounds have been chemically coupled to various probes like antibodies that will latch onto molecules of interest usually in fixed samples (immunofluorescence). SNAP-tag flagella will be suitable for such application. Furthermore, customers could purchase one aliquot of SNAP-tag flagella and then incubate them with particular fluorescent compounds to suit their specific needs. It will be easier to calculate molecule numbers by comparing identical fluorescing molecules. Immunofluorescence is the most common approach in fluorescent microscopy, especially in diagnostics. Reagents compatible with immunofluorescence will have a large market. 2B) Switch RSP3 to a different flagellar protein. The fluorescent intensity will increase if it periodicity is more frequent. Notably, once creating strains expressing one fluorescent fusion protein, we could cross them to recover the second generation that produces multi-color flagella with one protein carrying NeonGreen and the other protein carrying mCherry or SNAP tag. Such dual-tag standards will be suited for multi-color fluorescent microscopy. 2C) Break the fluorescent nanomachine into 9 fibers or 96-nm particle quants. Rather than intact flagella, individual fibers or particles with two NeonGreen molecules at 32-64 nm alternate periodicities could be used as a molecular ruler for super resolution microscopy and single molecule analysis whose popularity has exploded partly due to the recent Nobel Prize. Although DNA-based molecular rulers already exist for this purpose, we will explore if flagellar rulers have distinctive advantages, such as the production cost. 2D) Explore the application of the fluorescent standard in 3D imaging. So far we only test flagellar standards with small single cells. We likely need to modify applications for large samples of a wide focal plan range, like worms or zebrafish that typically requires different types of microscopes. Focal plans will have substantial effects on fluorescence intensities. We will test the standard with worms expressing GFP using the confocal microscope in our department and adjust quantification measures if deemed necessary. 2E) Develop packaging strategies that are compatible with commercialization. We would like to know the optimal way to package our products in ways that are convenient to customers and maximize the stability. Currently we have tested the liquid form. We are testing fixed flagella immobilized to glass slides. This may simplify the everyday usages in the diagnostic field.

### DISCUSSION

This study harnesses the defined RS periodicity and RSP3 stoichiometry in the 9+2 axoneme and Chlamydomonas RSP3 mutant to create flagella with fluorescent RSP3 of defined numbers. By imaging RSP3-NG flagella in various ways, this study showed its utility and limitation of fluorescent flagella as an intensity standard.

The ˜2× intensity of the overlapping regions as the non-overlapped region (FIG. 3) and the prorated intensities of axonemal sub-fibers and particles indicated a nearly 20× linear range from individual outer doublets to flagella with RSP3-NG. As the spectra and stability of NG and GFP are similar, RSP3-NG could be used as a standard for GFP fusion proteins, taking into consideration that different brightness of the two fluorophores.

However, several factors could deviate the estimates. For example, despite the reported NG's 2.7× brightness in comparison to EGFP, RSP3-NG flagella are 4× brighter than RSP3-GFP. One possible explanation is that the GFP sequence commonly used in Chlamydomonas is not identical to the sequences of the commonly used version. Or carrier proteins could affect FP intensity, either the neighboring tertiary molecular context, or the merely the size of carrier proteins. It is shown that molecular sizes inversely affect GFP intensity. It is unclear if the NG tagged to the ˜30-kD EB1 is brighter than the NG in ˜60-kD RSP3.

Parameters used during image acquisition matter. One is focal planes. Given the focal plane of widefield of fluorescence microscopy is about 200 nm, near the diameter of flagella, intensity of the out-of-focus region reduced substantially (FIG. 3A). The other is the degree of gain. We avoided to use gain to enhance image contrast at the expense of the linearity range. The other is excitation light intensity. Rapid photobleaching of GFP flagella was a reported concern. This is due to intense excitation light to compensate the weak GFP signal. This is less of a concern for NG of higher brightness. It is advisable to use minimal excitation light, by placing neutral filters in the light path, in exchange of a longer exposure time. This practice reduces photobleaching and phototoxicity as well. As shown in FIG. 5B, a 10-sec exposure did not hinder the subsequent 1 sec image acquisition. Once images are acquired, further adjustment of images will not affect profile plot analysis.

The brightness makes NG suitable for samples that need to methanol fixation. Contrary to a common misperception that methanol denatures FP, rendering invisible fluorescence, this study showed that ˜40% of RSP3-NG brightness was retained. This is not unique to NG or RSP3. EB1-NG at the flagellar tip could be acquired using a CCD camera after methanol fixation, although the intensity is very low (not shown). Methanol fixation is worth of considering in imaging objects that move rapidly or have strong autofluorescence. In Chlamydomonas, while methanol's effect in reducing autofluorescence does not compensate the reduced NG intensity, the treatment offers an independent assessment of EB1-NG comet intensity with reduced autofluorescence, more contrast images and immobilization of mobile cells that are challenging for image acquisition for a prolong exposure period.

The three examples showcase the new insight from estimating molecular abundance. It is well established that microtubule plus ends at the flagellar tip undergo constant turnover even in full length flagella with structures capping the plus ends. By comparing the abundance of EB1-NG at the flagellar tip and in comets, it becomes clear that EB1 plus tracking and thus tubulin turnover rate at the flagellar tip are relatively slow. As the tip-ward diffusion rate of EB1-NG is at the range of ˜10 μm/sec, the slow turnover is not due to the limited supply of EB1, but more likely due to the trafficking of dwindling GTP-tubulins (Craft et al., 2015) or the cap structures that may hinder tubulin turn over in full length flagella. With RSP3-NG fluorescent flagella as a standard, it only needs imaging quantification software to estimated numbers of unknown molecules. The similar dimension make estimate of fluorescent proteins in mitochondria using fluorescent flagella as a standard rather straightforward. While COX4-GFP has been used a marker to reveal altered dynamics of mitochondria in mutants, it is now feasible to estimate the numbers of mitochondrial proteins expressed under diverse conditions. Likewise, with flagellar standard, it is possible to estimate more accurately the changes in the abundance of Sis1and other chaperones and the misfolded proteins that they are trafficking at sub-cellular compartments at different temperatures and in different mutants.

This study uses RSP3-NG flagella and widefield epifluorescent microscopy to demonstrate the principle of flagellar fluorescence intensity marker. It may be applicable for confocal or deconvolution microscopy that acquire images in 3D. Flagellar makers built on the precise periodicity could be modified further, with carrier proteins of different periodicities and tag proteins of discreet properties. The utility of splayed outer doublets of RSP3-NG remains to be explored. The fluorescence of alternate 32 nm and 64 nm periodicity or the doublet particles could be potentially useful in superresolution or single molecule analysis. Given the ease of harvesting a large quantity of flagella from transgenic Chlamydomonas, it will be rather economic to apply fluorescent flagella intensity standard in everyday fluorescent imaging acquisition.

### REFERENCES

Bower, R., Tritschler, D., Vanderwaal, K., Perrone, C. A., Mueller, J., Fox, L., Sale, W. S., and Porter, M. E. (2013). The N-DRC forms a conserved biochemical complex that maintains outer doublet alignment and limits microtubule sliding in motile axonemes. Mol Biol Cell 24, 1134-1152.
Craft, J. M., Harris, J. A., Hyman, S., Kner, P., and Lechtreck, K. F. (2015). Tubulin transport by IFT is upregulated during ciliary growth by a cilium-autonomous mechanism. J Cell Biol 208, 223-237.
Craige, B., Brown, J. M., and Witman, G. B. (2013). Isolation of Chlamydomonas flagella. Curr Protoc Cell Biol Chapter 3, Unit 3 41 41-49.
Diener, D. R., Ang, L. H., and Rosenbaum, J. L. (1993). Assembly of flagellar radial spoke proteins in Chlamydomonas: identification of the axoneme binding domain of radial spoke protein 3. J Cell Biol 123, 183-190.
Ghaemmaghami, S., Huh, W. K., Bower, K., Howson, R. W., Belle, A., Dephoure, N., O'Shea, E. K., and Weissman, J. S. (2003). Global analysis of protein expression in yeast. Nature 425, 737-741.
Gupta, A., Diener, D. R., Sivadas, P., Rosenbaum, J. L., and Yang, P. (2012). The versatile molecular complex component LC8 promotes several distinct steps of flagellar assembly. J Cell Biol 198, 115-126.
Harris, J. A., Liu, Y., Yang, P., Kner, P., and Lechtreck, K. F. (2016). Single-particle imaging reveals intraflagellar transport-independent transport and accumulation of EB1 in Chlamydomonas flagella. Mol Biol Cell 27, 295-307.
Hom, E. F., Witman, G. B., Harris, E. H., Dutcher, S. K., Kamiya, R., Mitchell, D. R., Pazour, G. J., Porter, M. E., Sale, W. S., Wirschell, M., Yagi, T., and King, S. M. (2012). A unified taxonomy for ciliary dyneins. Cytoskeleton (Hoboken) 68, 555-565.
Huang, B., Piperno, G., Ramanis, Z., and Luck, D. J. (1981). Radial spokes of Chlamydomonas flagella: genetic analysis of assembly and function. J Cell Biol 88, 80-88.
Jensen, R. E., Hobbs, A. E., Cerveny, K. L., and Sesaki, H. (2000). Yeast mitochondrial dynamics: fusion, division, segregation, and shape. Microsc Res Tech 51, 573-583.
Kaganovich, D., Kopito, R., and Frydman, J. (2008). Misfolded proteins partition between two distinct quality control compartments. Nature 454, 1088-1095.
King, S. M., and Patel-King, R. S. (2015). The oligomeric outer dynein arm assembly factor CCDC103 is tightly integrated within the ciliary axoneme and exhibits periodic binding to microtubules. J Biol Chem 290, 7388-7401.
Marshall, W. F., and Rosenbaum, J. L. (2001). Intraflagellar transport balances continuous turnover of outer doublet microtubules: implications for flagellar length control. J Cell Biol 155, 405-414.
Michalet, X., Pinaud, F. F., Bentolila, L. A., Tsay, J. M., Doose, S., Li, J. J., Sundaresan, G., Wu, A. M., Gambhir, S. S., and Weiss, S. (2005). Quantum dots for live cells, in vivo imaging, and diagnostics. Science 307, 538-544.
Nillegoda, N. B., and Bukau, B. (2015). Metazoan Hsp70-based protein disaggregases: emergence and mechanisms. Front Mol Biosci 2, 57.
Norrander, J. M., deCathelineau, A. M., Brown, J. A., Porter, M. E., and Linck, R. W. (2000). The Rib43a protein is associated with forming the specialized protofilament ribbons of flagellar microtubules in Chlamydomonas. Mol Biol Cell 11, 201-215.
Oda, T., Yanagisawa, H., Yagi, T., and Kikkawa, M. (2014). Mechanosignaling between central apparatus and radial spokes controls axonemal dynein activity. J Cell Biol 204, 807-819.
Pedersen, L. B., Geimer, S., Sloboda, R. D., and Rosenbaum, J. L. (2003). The Microtubule plus end-tracking protein EB1 is localized to the flagellar tip and basal bodies in Chlamydomonas reinhardtii. Curr Biol 13, 1969-1974.
Pigino, G., Bui, K. H., Maheshwari, A., Lupetti, P., Diener, D., and Ishikawa, T. (2011). Cryoelectron tomography of radial spokes in cilia and flagella. J Cell Biol 195, 673-687.
Schmied, J. J., Raab, M., Forthmann, C., Pibiri, E., Wunsch, B., Dammeyer, T., and Tinnefeld, P. (2012). DNA origami-based standards for quantitative fluorescence microscopy. Nat Protoc 9, 1367-1391.
Schmied, J. J., Raab, M., Forthmann, C., Pibiri, E., Wunsch, B., Dammeyer, T., and Tinnefeld, P. (2014). DNA origami-based standards for quantitative fluorescence microscopy. Nat Protoc 9, 1367-1391.
Seetapun, D., Castle, B. T., McIntyre, A. J., Tran, P. T., and Odde, D. J. (2012). Estimating the microtubule GTP cap size in vivo. Curr Biol 22, 1681-1687.
Shaner, N. C., Lambert, G. G., Chammas, A., Ni, Y., Cranfill, P. J., Baird, M. A., Sell, B. R., Allen, J. R., Day, R. N., Israelsson, M., Davidson, M. W., and Wang, J. (2013). A bright monomeric green fluorescent protein derived from Branchiostoma lanceolatum. Nat Methods 10, 407-409.
Sivadas, P., Dienes, J. M., St Maurice, M., Meek, W. D., and Yang, P. (2012). A flagellar A-kinase anchoring protein with two amphipathic helices forms a structural scaffold in the radial spoke complex. J Cell Biol 199, 639-651.
Specht, S., Miller, S. B., Mogk, A., and Bukau, B. (2011). Hsp42 is required for sequestration of protein aggregates into deposition sites in Saccharomyces cerevisiae. J Cell Biol 195, 617-629.
Teves, M. E., Nagarkatti-Gude, D. R., Zhang, Z., and Strauss, J. F., 3rd. (2016). Mammalian axoneme central pair complex proteins: Broader roles revealed by gene knockout phenotypes. Cytoskeleton (Hoboken) 73, 3-22.
Vasudevan, K. K., Song, K., Alford, L. M., Sale, W. S., Dymek, E. E., Smith, E. F., Hennessey, T., Joachimiak, E., Urbanska, P., Wloga, D., Dentler, W., Nicastro, D., and Gaertig, J. (2015). FAP206 is a microtubule-docking adapter for ciliary radial spoke 2 and dynein c. Mol Biol Cell 26, 696-710.
Westermann, B. (2008). Molecular machinery of mitochondrial fusion and fission. J Biol Chem 283, 13501-13505.
Williams, B. D., Velleca, M. A., Curry, A. M., and Rosenbaum, J. L. (1989). Molecular cloning and sequence analysis of the Chlamydomonas gene coding for radial spoke protein 3: flagellar mutation pf-14 is an ochre allele. J Cell Biol 109, 235-245.
Wirschell, M., Zhao, F., Yang, C., Yang, P., Diener, D., Gaillard, A., Rosenbaum, J. L., and Sale, W. S. (2008). Building a radial spoke: flagellar radial spoke protein 3 (RSP3) is a dimer. Cell Motil Cytoskeleton 65, 238-248.
Yanagisawa, H. A., Mathis, G., Oda, T., Hirono, M., Richey, E. A., Ishikawa, H., Marshall, W. F., Kikkawa, M., and Qin, H. (2014). FAP20 is an inner junction protein of doublet microtubules essential for both the planar asymmetrical waveform and stability of flagella in Chlamydomonas. Mol Biol Cell 25, 1472-1483.

In the foregoing description, it will be readily apparent to one skilled in the art that varying substitutions and modifications may be made to the invention disclosed herein without departing from the scope and spirit of the invention. The invention illustratively described herein suitably may be practiced in the absence of any element or elements, limitation or limitations which is not specifically disclosed herein. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention. Thus, it should be understood that although the present invention has been illustrated by specific embodiments and optional features, modification and/or variation of the concepts herein disclosed may be resorted to by those skilled in the art, and that such modifications and variations are considered to be within the scope of this invention.

Citations to a number of references are made herein. The cited references are incorporated by reference herein in their entireties. In the event that there is an inconsistency between a definition of a term in the specification as compared to a definition of the term in a cited reference, the term should be interpreted based on the definition in the specification.

1. A fluorescent marker comprising a tubular or cylindrical biological structure formed by multiple copies of a structural protein (SP), the biological structure comprising multiple copies of a fluorescently-labeled protein (FP) regularly interspersed along the length of the biological structure.

2. The fluorescent marker of claim 1 comprising two FPs per 96 nm length of the biological structure.

3. The fluorescent marker of claim 1, wherein the structural proteins are assembled in a helical configuration.

4. The fluorescent marker of claim 1, wherein the structural protein comprises tubulin.

5. The fluorescent marker of claim 4, wherein the tubulin is α-tubulin (SEQ ID NO:6 or a variant thereof), β-tubulin (SEQ ID NO:7 or a variant thereof), or a combination of α-tubulin and β-tubulin as a heterodimer.

6. The fluorescent marker of claim 1, wherein the biological structure is a microtubule or a doublet microtubule.

7. The fluorescent marker of claim 1, wherein the biological structure is a proteinaceous microtubule or a proteinaceous doublet microtubule.

8. The fluorescent marker of claim 1, wherein the biological structure is a doublet microtubule comprising an A-microtubule and a B-microtubule.

9. The fluorescent marker of claim 1, wherein the fluorescently-labeled protein is a fusion protein comprising the amino acid sequence of a radial spoke protein (RSP) associated with a microtubule fused to the amino acid sequence of a fluorescent protein.

10. The fluorescent marker of claim 9, wherein the amino acid sequence of the fluorescent protein is fused to the C-terminus of the amino acid sequence of the RSP.

11. The fluorescent marker of claim 9, wherein: (a) the RSP is radial spoke protein 3 (RSP3) or a variant thereof that assembles into a microtubule structure comprising the amino acid sequence of SEQ ID NO:1 or a variant thereof having at least about 50%, 60%, 70%, 80%, 90%, 95%, 96%, 97%, 98%, or 99% sequence identity to SEQ ID NO:1; and (b) the fluorescent protein comprises the amino acid sequence of SEQ ID NO:2 or SEQ ID NO:3 or a variant thereof having at least about 50%, 60%, 70%, 80%, 90%, 95%, 96%, 97%, 98%, or 99% sequence identity to SEQ ID NO:2 or SEQ ID NO:3.

12. The fluorescent marker of claim 1, wherein the fluorescently-labeled protein is a fusion protein comprising the amino acid sequence of a radial spoke protein (RSP) fused to the amino acid sequence of an adapter protein that binds to a fluorescent label.

13. The fluorescent marker of claim 1, wherein the biological structure is an axoneme comprising multiple copies of the tubular or cylindrical biological structure.

14. A solid substrate comprising the fluorescent marker of claim 1 immobilized to the solid substrate.

15. A method for performing fluorescence microscopy, the method comprising detecting fluorescence from the fluorescent marker of claim 1 and detecting fluorescent from a fluorescent sample.

16. The method of claim 15, wherein the fluorescent marker is immobilized to a solid substrate.

17. A fusion protein comprising the amino acid sequence of a radial spoke protein (RSP) associated with a microtubule fused to the amino acid sequence of a fluorescent protein.

18. A fusion protein comprising the amino acid sequence of a radial spoke protein (RSP) fused to the amino acid sequence an adapter protein that binds to a fluorescent label.

19. A polynucleotide encoding the amino acid sequence of the fusion protein of any of claim 17.

20. A polynucleotide encoding the amino acid sequence of the fusion protein of any of claim 18.


SEQUENCE LISTING (8 sequences)

SEQ ID NO: 1 - protein, 516 aa, Chlamydomonas reinhardtii
MVQAKAQQQLYTHAAEPKAVQQRRAKYREDETTQTLPTANIMFDRRVVRGNTYAARILPADATQTQTKGPSPASTKKRTTRTLPPRTPEAVDGRRHIDIQTDVYLEELTDTVPEADTSTQTDAFLDRPPTPLFVPQKTGTDAITQIENGDLFDFDFEVEPILEVLVGKVLEQGLMEVLEEEELAAMRAHQEHFEQIRNAELVATQRMEAAERRKLEEKERRMQQERERVERERVVRQKVAASAFARGYLSGIVNTVFDRLVSSGYIYDPVMREVETAFMPWLKEQAIGYLARGVVARRVVDKLVEDAAAALAANRSTLADKAASTAATVDAWAERQAKMEAELQGKELEAVRRRPTFVLRELKPAVASADAVEAAAAELTAQAEEAANAKWEADKAEAAEKARAEAEAAAEEQKALLEELAATAAAEAEERGEEPPAEPPSLPDGVEPVDVEAEVAKAVEAVPKPPVKEVTDIDILSYMMDKGAITKDAIIQALAVHALGDKAYTNHPAFAEAEGA

SEQ ID NO: 2 - protein, 244 aa, Aequorea victoria
MASLADPPKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSIQSALSKDPNEKRDHMVLLEFVTAAGITHGMDELYK

SEQ ID NO: 3 - protein, 236 aa, Branchiostoma lanceolatum
LVQLVHAHHVGERLLPLLEVQLRLAVLQLGLAEHVHRLVLQVVRGHGLRERVRGAGGRAVALAVARGVRPLERADDGLVVGVGLLGAAPVRGRQRVGHHRAVRGEAGALHLGLALDVAALVRVAVVHGQGGAVLELHGAVHLVARAVHHRGLERAHAVRVGQVLVEAVADVRHQNPGAELQVALRGLQVQLLVAVVGVAGALAHHVEVHAVDAAEDVQLVRRGQARHVVLLALGHH

SEQ ID NO: 4 - protein, 755 aa, Artificial
  Fusion protein of Chlamydomonas reinhardtii RSP3 and Aequorea victoria GFP
MVQAKAQQQLYTHAAEPKAVQQRRAKYREDETTQTLPTANIMFDRRVVRGNTYAARILPADATQTQTKGPSPASTKKRTTRTLPPRTPEAVDGRRHIDIQTDVYLEELTDTVPEADTSTQTDAFLDRPPTPLFVPQKTGTDAITQIENGDLFDFDFEVEPILEVLVGKVLEQGLMEVLEEEELAAMRAHQEHFEQIRNAELVATQRMEAAERRKLEEKERRMQQERERVERERVVRQKVAASAFARGYLSGIVNTVFDRLVSSGYIYDPVMREVETAFMPWLKEQAIGYLARGVVARRVVDKLVEDAAAALAANRSTLADKAASTAATVDAWAERQAKMEAELQGKELEAVRRRPTFVLRELKPAVASADAVEAAAAELTAQAEEAANAKWEADKAEAAEKARAEAEAAAEEQKALLEELAATAAAEAEERGEEPPAEPPSLPDGVEPVDVEAEVAKAVEAVPKPPVKEVTDIDILSYMMDKGAITKDAIIQALAVHALGDKAYTNHPALEMASLADPPKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSIQSALSKDPNEKRDHMVLLEFVTAAGITHGMDELYK

SEQ ID NO: 5 - protein, 747 aa, Artificial
  Fusion protein of Chlamydomonas reinhardtii RSP3 and Branchiostoma lanceolatum mNeonGreen Protein
MVQAKAQQQLYTHAAEPKAVQQRRAKYREDETTQTLPTANIMFDRRVVRGNTYAARILPADATQTQTKGPSPASTKKRTTRTLPPRTPEAVDGRRHIDIQTDVYLEELTDTVPEADTSTQTDAFLDRPPTPLFVPQKTGTDAITQIENGDLFDFDFEVEPILEVLVGKVLEQGLMEVLEEEELAAMRAHQEHFEQIRNAELVATQRMEAAERRKLEEKERRMQQERERVERERVVRQKVAASAFARGYLSGIVNTVFDRLVSSGYIYDPVMREVETAFMPWLKEQAIGYLARGVVARRVVDKLVEDAAAALAANRSTLADKAASTAATVDAWAERQAKMEAELQGKELEAVRRRPTFVLRELKPAVASADAVEAAAAELTAQAEEAANAKWEADKAEAAEKARAEAEAAAEEQKALLEELAATAAAEAEERGEEPPAEPPSLPDGVEPVDVEAEVAKAVEAVPKPPVKEVTDIDILSYMMDKGAITKDAIIQALAVHALGDKAYTNHPALELVQLVHAHHVGERLLPLLEVQLRLAVLQLGLAEHVHRLVLQVVRGHGLRERVRGAGGRAVALAVARGVRPLERADDGLVVGVGLLGAAPVRGRQRVGHHRAVRGEAGALHLGLALDVAALVRVAVVHGQGGAVLELHGAVHLVARAVHHRGLERAHAVRVGQVLVEAVADVRHQNPGAELQVALRGLQVQLLVAVVGVAGALAHHVEVHAVDAAEDVQLVRRGQARHVVLLALGHH

SEQ ID NO: 6 - protein, 451 aa, Chamydomonas reinhardtii
MREVISIHIGQAGIQVGNACWELYCLEHGIQPDGQMPSDKTIGGGDDAFNTFFSETGAGKHVPRCIFLDLEPTVVDEVRTGTYRQLFHPEQLISGKEDAANNFARGHYTIGKEIVDLALDRIRKLADNCTGLQGFLVFNAVGGGTGSGLGSLLLERLSVDYGKKSKLGFTVYPSPQVSTAVVEPYNSVLSTHSLLEHTDVAVMLDNEAIYDICRRSLDIERPTYTNLNRLIAQVISSLTASLRFDGALNVDITEFQTNLVPYPRIHFMLSSYAPIISAEKAYHEQLSVAEITNAAFEPASMMVKCDPRHGKYMACCLMYRGDVVPKDVNASVATIKTKRTIQFVDWCPTGFKCGINYQPPTVVPGGDLAKVQRAVCMISNSTAIGEIFSRLDHKFDLMYAKRAFVHWYVGEGMEEGEFSEAREDLAALEKDFEEVGAESAEGAGEGEGEEY

SEQ ID NO: 7 - protein, 443 aa, Chamydomonas reinhardtii
MREIVHIQGGQCGNQIGAKFWEVVSDEHGIDPTGTYHGDSDLQLERINVYFNEATGGRYVPRAILMDLEPGTMDSVRSGPYGQIFRPDNFVFGQTGAGNNWAKGHYTEGAELIDSVLDVVRKEAESCDCLQGFQVCHSLGGGTGSGMGTLLISKIREEYPDRMMLTFSVVPSPKVSDTVVEPYNATLSVHQLVENADECMVLDNEALYDICFRTLKLTTPTFGDLNHLISAVMSGITCCLRFPGQLNADLRKLAVNLIPFPRLHFFMVGFTPLTSRGSQQYRALTVPELTQQMWDAKNMMCAADPRHGRYLTASALFRGRMSTKEVDEQMLNVQNKNSSYFVEWIPNNVKSSVCDIPPKGLKMSATFIGNSTAIQEMFKRVSEQFTAMFRRKAFLHWYTGEGMDEMEFTEAESNMNDLVSEYQQYQDASAEEEGEFEGEEEEA

SEQ ID NO: 8 - protein, 190 aa, Chlamydomonas reinhardtii
MFKNAFQSGFLSVLYSIGSKPLEIWDKQVSNGHIKRITDADIQSSVLEIMGQNVSTTYITCPADPNKTLGIKLPFLVLIIKNLNKYFSFEVQVLDDKNVRRRFRASNYQSTTRVKPFICTMPMRLDSGWNQIQFNLSDFTRRAYGTNYIETLRVQVHANCRIRRIYFSDRLYSEEELPAEFKLFLPIQKS